(19) United States
(12) Patent Application Publication
(10) Pub. No.: US 2026/0027267 A1
(43) Pub. Date: Jan. 29, 2026

(54) Nitric Oxide Loaded Articles

(71) Applicant: University Court of the University of St Andrews, Fife (GB)

(72) Inventors: Stewart Warrendar, Kennoway (GB); Morven Duncan, Dundee (GB); Russell Morris, Gauldry (GB)

(21) Appl. No.: 19/271,073

(22) Filed: Jul. 16, 2025

Related U.S. Application Data
(60) Provisional application No. 63/674,998, filed on Jul. 24, 2024.

(51) Int. Cl. A61L 29/06; A61L 29/16; B01J 20/22; B01J 20/30

(52) U.S. Cl. CPC A61L 29/06; A61L 29/16; B01J 20/226; B01J 20/3071; A61L 2300/114

(57) ABSTRACT

The present disclosure is concerned with articles for releasing adsorbed NO gas, wherein the articles comprise a metal-organic framework (MOF)/polymer composite material to which the gas is releasably adsorbed. The article may be in the form of a medical article, or portion thereof, for insertion into a body, for example. There is further taught methods of adsorbing and releasing a gas from the article/medical article.

### CROSS-REFERENCE TO RELATED APPLICATIONS

The present application claims the benefit of U.S. Provisional Patent Application No. 63/674,998, filed on Jul. 24, 2024, which is incorporated herein by reference.

### FIELD

The present disclosure is concerned with articles for releasing adsorbed Nitric Oxide (NO) gas, wherein the articles comprise a metal-organic framework (MOF)/polymer composite material to which the gas is releasably adsorbed. The article may be in the form of a medical article, or portion thereof, for insertion into a body, for example. There is further taught methods of adsorbing and releasing a gas from the article/medical article.

### INTRODUCTION

Metal-organic frameworks (MOFs) are one of the most significant classes of materials developed in recent times. Formed by connecting metal ions or clusters with organic molecules, their extended, three-dimensional network structures possess high porosities and surface areas (up to 6,000 m² g⁻¹), and demonstrate almost unparalleled tunability in their physicochemical properties. The large surface area makes MOFs attractive for high-capacity gas adsorption and storage applications, with potential for hydrogen storage, the adsorption of water from desert air and the large-scale capture of carbon dioxide from flue gases. MOFs have also been extensively studied for their bio-medical applications⁶, with a focus on drug storage and delivery.

The challenges when designing materials that can be used in medical devices are significantly different from those associated with other adsorption-based applications. Rather than a balance between high capacity, regeneration and durability as is the case for MOF applications listed above, for medical device applications it is the balance between controlled, consistent delivery (dosage), toxicology and suitable form of the material that is important. NO is a gaseous biological signalling molecule. Among its many attributes it is a potent antimicrobial agent with activity against biofilms, triggers vasodilation and prevents thrombus formation. Exogenous delivery has the potential to offer many varied advanced therapies that mimic natural processes and address pressing healthcare challenges such as catheter-related infection and thrombosis. Targeted NO function depends on precise delivery at the correct concentration for the desired effect. Localised and controlled delivery, e.g. from indwelling devices, has long been sought but is yet to be realised. Previous studies have demonstrated the high capacity of MOFs to store NO, and their ability to release their payloads under humid conditions at biologically active concentrations¹, to provide vasodilatory², antithrombotic³ and antibacterial⁴ activity. However, the major challenge of how to formulate MOFs into useable materials or devices while retaining their activity has not been solved.

Despite major progress in the development of MOFs in recent years, and despite some notable examples of MOF-based products nearing or reaching commercialisation⁵, the challenge of translating from lab to marketable product persists. A particular aspect of this challenge is the shaping and post processing of MOF powders into suitable form for a given application without loss of function—the development of functionally appropriate formulations is the key challenge facing all MOF applications, not just those associated with biomedicine. Techniques have been developed and tested to shape MOF powders into pellets, granules, beads etc., which are suitable and necessary for applications such as gas adsorption/storage and catalysis⁶⁻⁸. The creation of MOF composites, such as through the combination of MOFs with polymers to create mixed matrix membranes, is another approach to create suitable materials for gas separation and capture⁹. For other applications, MOFs will need to be processed and incorporated into three-dimensional end products, many of which are traditionally made from polymers and are typically produced using extrusion and/or injection moulding processes. The combination of a MOF embedded within a porous polymer matrix means that control over gas delivery profiles is significantly more challenging than from the MOFs alone.

Most shaping studies appear to focus on pellets, granules and membranes, and very few studies exist that consider using melt processing to incorporate MOFs into polymer-based shaped articles, such as tubes. In view of the breadth of research being conducted in the field of MOFs, it is surprising that very little has been reported in this area, and only two MOFs appear to have been studied. HKUST-1 was extruded into thermoplastic polyurethane tubing to catalyse the generation of NO from bioavailable S-nitrosothiols¹⁰. The prepared tubing successfully catalysed NO release, though no analysis of the influence of the MOF on the polymer and its properties was reported. Melt extrusion was more recently employed in the preparation of ZIF-8-containing polypropylene test pieces in a study analysing the effectiveness of the MOF as a synergist for intumescent flame retardants. In other studies, ZIF-8 and HKUST-1 were processed via melt extrusion/injection moulding into poly L-lactic acid to assess their influence on mechanical and barrier properties¹⁻¹⁴. HKUST-1 was also incorporated into poly lactic acid by melt extrusion to form granules for the removal of mercury from water.¹⁵ Reports in these studies that MOF-derived moisture can adversely impact both MOF and polymer during melt processing, and analysis suggesting MOFs can both positively and negatively impact physicochemical properties of the polymers after compounding, indicate that processing in this way is not trivial.

### SUMMARY

In a first aspect there is provided an article comprising at least a portion for releasing adsorbed NO gas, the portion comprising, consisting essentially of, or consisting of, a polymer/metal organic framework (MOF) composite material, wherein the MOF is partially activated and NO gas is bound to (typically within pores of) the MOF structure.

As identified and described further herein, the present inventors have identified that unexpectedly, partial activation may result in NO being bound to water molecules that remain bound within the MOF structure following partial activation. Thus, in one teaching, partial activation leads to NO being bound at least to water molecules remaining in the MOF following partial activation. In one teaching, NO gas is bound to both metal and water molecules present within pores of the MOF.

As will be further described herein, partial activation involves the partial or incomplete removal of water molecules from the MOF structure, as compared to full activation, which removes substantially all water from the MOF. As such, partial activation means that a significant degree of water occupancy is still present within the MOF, typically between 15-60%, such as 20-50%, of full or complete water occupancy. Unexpectedly, the present authors have discovered that following partial activation NO is able to bind to exposed metal sites and also to a metal site comprising a water molecule that has not been removed during the partial activation process.

The composite material of the present disclosure comprises a bulk polymer with a MOF present within it. For example, the MOF may be processed from a powder and may be present in the form of beads, pellets, granules, or the like that are dispersed within the polymer. The MOF may be present within the polymer in an amount of between 0.1-12 wt %, 0.1/0.25-10 wt %, such as 0.1/0.25/0.5-5 wt %.

The polymer may be a hydrophilic or hydrophobic polymer or copolymer. A copolymer is a polymer that is made up of two or more monomer species. Use of a hydrophilic polymer may facilitate water ingress within the polymer/copolymer of the composite material and contact with the MOF dispersed therein.

The polymer may be a thermoplastic polymer or copolymer, such as a thermoplastic elastomer. A thermoplastic is any plastic polymer material that becomes pliable or moldable at a certain elevated temperature and solidifies upon cooling. For example, the polymer may be a polyether; polyurethane; polyamide; polyolefin; polyvinyl chloride; polybutadiene; suitable thermoplastic fluoropolymers, such as perfluoroalkoxy (PFA); neoprene; or silicone polymer or co-polymer.

In one teaching, the polymer may be a block copolymer. A block copolymer may be made up of blocks of different polymerised monomers. In one example, the block polymer comprises blocks of rigid and soft blocks. An exemplary class of block copolymers are polyether/polyamide co-polymers (PEBA), which include copolymers sold under the PEBAX® and VESTAMID® tradenames.

In one teaching, the composite material is flexible and may optionally be in the form of a tube. For medical applications, such tubes may, although not exclusively, be used for insertion into the body (such as into a vessel, organ, or tissue) and for the delivery or removal of fluids to/from the body. Examples of such tubes, include catheters and cannulas, although these terms may be used interchangeably and introducer sheaths. In a particular embodiment, the tube, such as a catheter, cannula, or introducer sheath, is intended to be retained for an extended period of time within the body—by extended period of time, it is understood that this refers to at least one hour, two or more hours, at least a day, two or more days, at least a week, or two or more weeks, before being removed from the body. For catheters, catheters that are retained in place for an extended period of time, may be called an indwelling or suprapubic catheter. Cannulas may be understood as indwelling single-lumen conduits that allow fluids, medications and other therapies such as blood products to be introduced directly into, for example, a peripheral vein.

In one teaching, the article as described herein, may have been prepared by a melt extrusion process. A suitable melt extrusion process may comprise melting the polymer within a mixture comprising polymer beads, particles, powder or the like and MOF particles or powders, through a combination of applied heat and friction. Prior to processing, MOFs typically comprise significant amounts of water. However, for extrusion, it is desirable for polymers to have a very low water content. Thus, as well as ensuring the polymer is low in water content, it is necessary to dry the MOF, in order to remove water. Desirably, less than 10 wt % water is present in the MOF, prior to co-extrusion with the chosen polymer.

For extrusion, typically, the melting temperature must be sufficient to melt the polymer, but not the MOF. Prior to melting and/or extrusion, the polymer and MOF components may be dried and kept under a dry inert gas, such as nitrogen, in order to remove water from the polymer and MOF components and prevent its readsorption during the extrusion process. This molten polymer/MOF particles or powder mixture is then forced under high pressure through a small orifice/die or, for example, a “shower head” of orifices/dies called a spinneret. The molten polymer/MOF stream flowing out of each orifice solidifies into a solid product at some distance from the orifice. The solidified product may be reheated and drawn numerous times as the product traverses the extrusion line to the final product or article. Alternatively, once the melt has come through the die—there is tension on the extrudate to draw to the correct dimensions and very quickly (within cm of the die) the tube is plunged into a water bath in order to solidify. In such a process, there is no reheating to draw to the correct dimensions.

The resulting extruded material comprises an extruded polymer with MOF dispersed throughout.

MOFs, as used herein, are a class of crystalline porous materials, in which metal ions (Mⁿ⁺) or clusters of metal ions are linked together with linkers (L^{y-}) to form three dimensional networks, defining extended pores and channels of molecular dimension. The channel networks may extend in one, two or three dimensions. The channel networks may intersect and may define internal cavities. Metal organic frameworks may therefore be regarded as materials, which define a large internal (and external) surface area. MOFs may be regarded as a class of zeotype materials.

The MOF may comprise a single type of metal ion, or more than one type of metal ion. The MOF may comprise metal ions of one or more transition metals, alkali metals, alkaline earth metals and/or other suitable metal cations, such as for example aluminium. The MOF may comprise ions of a metallic element in more than one oxidation state.

The MOF may comprise one or more framework transition metal ions selected from (but not limited to): Tiⁿ⁺, Vⁿ⁺, Crⁿ⁺, Mnⁿ⁺, Feⁿ⁺, Coⁿ⁺, Niⁿ⁺, Cuⁿ⁺, Znⁿ⁺, Agⁿ⁺, Ru, Rh where n is 1, 2, 3 or 4, depending on the metal and the oxidation state of that metal.

The MOF may comprise one or more framework transition metal ions selected from: Cu⁺, Cu²⁺, Mn²⁺, Mn³⁺, Zn²⁺, Fe²⁺, Fe³⁺, V³⁺, V⁴⁺, Ag⁺, Ru³⁺, Rh³⁺, Ni²⁺, Cr²⁺, Co²⁺ and Co³⁺. For example, the framework metal ion(s) may be selected from Cu⁺, Cu²⁺, Cr²⁺, Zn²⁺, Co²⁺, Co³⁺, Ag⁺, Ni²⁺, Mn²⁺ and Mn³⁺. Preferably the framework metal ion(s) are selected from Cu²⁺, Zn²⁺, Ag⁺, Ni²⁺, Mn²⁺.

The MOF may comprise one or more framework alkali metal ions selected from, for example: Na⁺ and K⁺.

The MOF may comprise one or more framework alkaline earth metal ions selected from (but not limited to) Ca²⁺ and Mg²⁺, especially Mg²⁺.

Other framework metal ions, which may be present (alternatively or in addition to the above) may include for example Al³⁺.

In some embodiments (e.g. for medical applications), it may be preferred for the metal ions present in the MOF to be those which are deemed toxicologically acceptable for such uses, e.g. those metals which are considered to have acceptable/limited toxicity. Such considerations will depend on the circumstances of the use and may be determined by the skilled practitioner as appropriate. For example, the ions of Mg, Ca, Fe or Mn may be considered to have low toxicity. A balance may exist between acceptable toxicity and antimicrobial efficacy. For example Ag is known to demonstrate antimicrobial properties and has acceptable toxicity, as to the ions of Ni, Cu and Zn (which is less toxic but generally less antimicrobially active).

The MOF may comprise a single type of ligand, or more than one type of ligand. MOFs comprising more than one type of ligand and/or metal may be referred to as “mixed component” MOFs, or more specifically mixed-ligand or mixed-metal MOFs.

Each ligand may include one coordinating functional group, or more than one coordinating functional group. A coordinating functional group may comprise one atom or more than one atom which coordinates to a given metal ion.

For example, each ligand may include 2-10 coordinating sites, e.g. 2-6 coordinating sites, or 2-4 coordinating sites; for example 2 or 3 coordinating sites.

The MOF may comprise a polydentate ligand, for example a bidentate, tridentate or a ligand having another order of denticity.

The MOF may comprise carboxylate or polycarboxylate ligands, for example, benzene polycarboxylate ligands. A polycarboxylate ligand may be a polydentate (for example di- or tridentate) linking ligand.

A benzene polycarboxylate ligand may comprise a benzene ring and at least two carboxylate groups and, optionally, one or more further substituents to the benzene ring.

The MOF may be any suitable MOF with pore size large enough (such as greater than 4 angstroms in the smallest dimension) to accommodate gas molecules.

The MOF may comprise for example 1,4-benzenedicarboxylate, 1,3,5-benzene tricarboxylate (BTC), dihydroxy benzene dicarboxylate, in particular 2,5-dihydroxyterphthalate (DHTP) and/or the like.

The MOF may be, for example, MOF-74 (CPO-27), HKUST-1, STAM-1, MIL101 and SIP-3.

The MOF may comprise more than one type of ligand.

The MOF may comprise or contain additional entities to those described above, for example, further metal or other positively charged ions, or other anionic species.

Further anions may include halogens, e.g. Cl⁻, F⁻, Br⁻ or I⁻ or other anions, e.g. OH⁻ or SO₄⁻.

The metal organic frameworks may in particular include species/molecules, within guest sites, such as pores or channels, formed in the framework. Such species may be for example water, solvent or other molecules e.g. derived from the components used in the manufacture of the framework.

One or more of the water molecules may be present as hydrating water molecules and be bound to the network structure, for example to a framework metal ion or a framework ligand.

As will be understood by a skilled reader, one or more water molecules may be disassociated, for example so as to form a protonated “H₃O⁺” species and a coordinating OH⁻ ligand, together with a further water molecule.

The total amount of water may vary depending on the degree of hydration of the MOF, for example due to variations in ambient humidity, temperature, contact with biological fluids, etc.

Hydroxide ligands may form part of the framework structure, and may be coordinated to more than one metal ion within the framework structure.

The invention is not limited to a particular MOF morphology or structural type. The MOF may for example be of the structural type STAM-1, CPO-27 or HKUST-1, the synthesis and properties of which are generally described in WO 2008/020218, WO 2012/020214 and WO 2013/186542, to which the skilled addressee is directed. The MOF may be of the structural type SIP-3.

In accordance with the teaching provided herein, the NO gas may be both chemisorbed and physisorbed within pores of the MOF. Adsorption caused by electrostatic and/or Van der Waals forces, is referred to as physical adsorption or physisorption, whereas if a chemical bond is formed between the gas and the MOF, this is referred to as chemical adsorption or chemisorption. NO gas may also be physisorbed by the polymer of the polymer/MOF composite material.

The NO gas may interact, including chemically or physically binding to framework metal ions, framework ligands and/or other extra-framework species such as water, or extra-framework cations or anions of the MOF. As mentioned above and further described herein, the present authors have unexpectedly discovered that NO gas is able to bind strongly to metal and water molecules present in a partially activated MOF.

NO gas may defuse out of the MOF and/or polymer over the course of time. The gas may be capable of being displaced by another species such as water or another small molecule on exposure of the MOF thereto. Alternatively or in addition, the NO gas may be capable of being released from the MOF responsive to a stimulus, such as a change in temperature or pressure. The rate of release of the gas from the MOF may be altered (e.g. accelerated) by a stimulus.

The article may find application in medical and non-medical fields. In one embodiment of the present disclosure, the article is in the form of a medical article, such as for insertion within the body, or a body cavity or orifice of a subject, such as a human or animal subject.

Advantageously, the articles or medial articles described herein, may release NO gas over an extended period of time. When in the form of a medical article, the polymer/MOF composite material may desirably release the NO gas whilst the medical article, or portion thereof comprising the polymer/MOF composite material, is present within a subject. The extended period of time may be similar to the extended period of time the medical article is present within the body of a subject. Thus, the extended period of time that the medical article releases the NO gas may be at least one hour, two or more hours, 6, 12 or 24 hours, or even days. In this manner NO gas may be released within the subject for at least some of the time, or substantially all of the time, the medical article or portion thereof, is present or dwelling within the body.

In one embodiment, the article is a medical article that comprises, nitric oxide (NO). As well as having antimicrobial properties, NO also, when administered in suitable amounts, can have other beneficial physiological properties. For example, and as demonstrated herein, NO can be delivered in order to trigger vasodilation by inducing vascular relaxation; and prevent thrombus formation by reducing platelet aggregation. In order to provide one or more of the aforementioned medically useful properties (antimicrobial effect, vascular relaxation and/or reducing platelet activation), the medical article in accordance with the present disclosure, is capable of delivering NO in a therapeutic range of approximately 1 pmol to 100 μmol. For indwelling articles, such as catheters and cannulas, release of NO from the catheter or cannula may be particularly advantageous in terms of the therapeutic effects NO may provide in the locality of where the catheter/cannula is located. Moreover, the aforementioned medically useful properties are provided locally, as opposed to systemically, which may be further advantageous.

Moreover, medical articles in accordance with the present disclosure may be non-toxic, or considered to possess minimal toxicity, for at least the period of time in which the medical article is being used/in contact with the body.

Previous wisdom may have suggested that the most efficient way of loading a gas into a MOF, would be to first completely dehydrate or dry the MOF, in order to remove all or substantially all water molecules from the MOF and thereafter allow the gas, such as NO, to adsorb into the MOF. However, it has now been found, as described herein, that a partial activation, whereby only a portion of water molecules are removed from the MOF, still allows sufficient NO gas to adsorb into the MOF, for subsequent release. Moreover, as observed, following such partial activation, a gas becomes bound to both metal and water molecules, leading to more NO being adsorbed than would have been expected following partial activation. Without wishing to be bound by theory, as a gas becomes both chemisorbed and physiosorbed within a MOF, the gas can be released from the MOF by both passive and active processes, thereby permitting a gas to be released from the MOF over an extended period of time.

A further advantage of conducting only a partial activation, is that lower temperatures than conventionally used for full activation, such as equal to or less than 90° C., 80° C., 70° C., 60° C., or even 50° C. may be used. In any case, a temperature, for partial activation, may be chosen that is lower than the melting point and that is outside the breadth of the glass transition, or the temperature range over which a material changes from a glassy to a rubbery material. The skilled addressee is aware that the glass transition temperature range varies depending on the polymer employed and there are published tables identifying the glass transition temperature of various polymers. There are a variety of thermal and mechanical analytical techniques that can be used to measure the glass transition temperature (Tg). Most notably these include: Differential Scanning Calorimetry (DSC), Dynamic Mechanical Analysis (DMA) and Thermomechanical Analysis (TMA) depending on the sample. Such temperatures, lower than may have been conventionally employed for full activation of a MOF, have been found to be suitable to partially activate the MOF and enable significant amounts of NO to be bound, whilst ensuring that the polymer itself does not melt. Conventional wisdom may have suggested that lower activating temperatures that lead to only partial activation of the MOF, whilst being suitable in terms of not melting the polymer of the composite material, would not allow sufficient NO to bind and be available for subsequent release for a suitable use, such as a medical use.

As well as heating the composite at a temperature that is lower than the melting point and sufficiently above or below the glass transition temperature of the polymer of the composite material, the partial activation method may comprise exposing the composite to sub atmospheric pressure (for example of the order of less than 10⁻², 10⁻³ or 10⁻⁴ Torr).

In order for a gas to be adsorbed within the composite, the composite comprising the partially activated MOF may be exposed to one or more atmospheres (e.g. 2, 3 4, or 5 bar, for example) of NO gas.

The composite comprising the partially activated MOF may be contacted with NO gas for any suitable period of time, which may vary for example depending on the composition, purity or morphology of the MOF and/or polymer. Typically, for example, contacting with NO gas may take place over the course of around an hour, but can be shorter or longer as required.

The skilled addressee will appreciate that the optimal conditions and duration of the steps may be determined by monitoring the progress of the reaction (for example by monitoring changes in pressure, gravimetric analysis, spectroscopically, and so forth).

Excess and/or weakly bound NO gas may be removed by purging the composite. A suitable purge regime comprises several (e.g 1-3) cycles of applied vacuum (sub atmospheric pressure, such as 10⁻² torr, for example) and flushing with an inert gas, such as argon. The composite may further be treated to a stringent purging process for a period to allow the NO release profile to stabilise Stringent purging may involve placing the article under a vacuum (sub atmospheric pressure, as above) at a temperature above room temperature. A stringent purge regime comprises exposing the composite with adsorbed NO gas, to a reduced vacuum for several hours (e.g. 2-24, such as 4-6 hours) at an elevated temperature (e.g. 40° C.-60° C., such as 50° C.). It has been observed that following such stringent purge methods, a more consistent gas release profile from the composite material is observed. Without this stringent purge process, there is generally an initial burst release of gas from the composite, which may be undesirable for certain applications, including medical applications, where a more consistent release may be preferable.

Without wishing to be bound by theory, it is proposed that a significant component of NO gas released from MOF-polymer composites treated in a conventional way (and particularly that which contributes to the initial burst) does not originate from the observed metal and water NO binding sites in the MOF. It is proposed that gas is additionally present within the composites as physisorbed molecules that are weakly held in cavities and voids in the polymer matrix, and/or associated with external surfaces of the composite and/or MOF particles within the composite. It is considered that such weakly bound gas contributes to the instability of the release performance with storage. Thus, it has been found that by applying a stringent purging process described above, some of the weakly bound physisorbed gas is removed from the system leaving a greater proportion of more tightly bound gas. This allows a more consistent gas release profile to be observed.

In order to release bound NO gas from the composite material, the composite material comprising bound NO gas may be contacted with an aqueous environment, such as water or blood. NO gas is displaced from the MOF, resulting in release of NO gas into the aqueous environment.

As well as articles/medical articles described herein that comprise NO gas bound thereto, the present disclosure extends to such articles/medical articles, prior to contacting with NO gas.

There is further provided, a method of treating a subject in need thereof comprising providing a medical article, as described herein and contacting at least a portion of the composite material comprising bound NO with blood or an aqueous environment found within the body of the subject, in order to release NO.

There is further provided a medical article, as described herein, for use in a method of treating a subject.

The release of NO from the medical article may provide an antimicrobial effect, vascular relaxation and/or reducing platelet activation.

The present invention will now be further defined by the following numbered clauses:
1. An article comprising at least a portion for releasing adsorbed NO gas, the portion comprising, consisting essentially of, or consisting of, a polymer/metal organic framework (MOF) composite material, wherein the MOF is partially activated and comprises NO gas bound to the MOF structure.
2. An article according to clause 1, wherein the article is a medical article for releasing a physiological beneficial amount of the adsorbed NO gas over a period of time into a body of a subject.
3. The article according to clauses 1 or 2, wherein the NO gas is bound to at least water molecules bound to the MOF following partial activation, or both metal and water molecules present within pores of the MOF and optionally physisorbed NO that is bound within the polymer matrix.
4. The article according to any preceding clause, wherein the MOF is present in the composite material in the form of particles or powder, dispersed within the polymer.
5. The article according to any preceding clause, wherein the MOF is present in the composite material in an amount of between 0.1-12 wt %, 0.1/0.25-10 wt %, such as 0.1/0.25/0.5-5 wt %.
6. The article according to any preceding clause, wherein the MOF comprises a CPO-27-M framework, wherein M═Co, Fe, Mn, Mg, Ni, or Zn, such as Ni or Zn.
7. The article according to any preceding clause, wherein the polymer is a hydrophilic or hydrophobic polymer or copolymer.
8. The article according to any preceding clause wherein the polymer is a thermoplastic polymer or copolymer, such as a thermoplastic elastomer.
9. The article according to clause 8, wherein the thermoplastic polymer is a polyether, polyurethane, polyamide, polyolefin, a fluoropolymer, polybutadiene, polyvinyl chloride, neoprene, or silicone polymer or co-polymer, comprising one or more of the identified polymers.
10. The article according to clause 9 wherein the polymer is a block copolymer comprising of rigid and soft blocks.
11. The article according to clause 10, wherein the block copolymer is a polyether/polyamide co-polymer (aka PEBA), such as a Pebax® or Vestamid® polymer.
12. The article according to clauses 9-11 wherein the composite material is rigid, partially flexible, or flexible.
13. The article according to any preceding clause, wherein the composite material has been prepared by a melt process.
14. The article according to any preceding clause, wherein the composite material is in the form of a tube.
15. A medical article according to clause 14, when dependent on claim 2, wherein the tube is part of a medical catheter, cannula, or introducer sheath for insertion within a body, vessel, or organ of a subject, or is suitable for medical uses that do not require insertion into a body.
16. The medical article according to clause 15 wherein the tube is intended be retained within the body, vessel, or organ of a subject for a period of between 10 min, 20 min, or 30 min—14 days, such as 1 hour to 2 days, or 2 hours to 24 hours—1 week.
17. A method of adsorbing a NO gas within a polymer/MOF composite material, the method comprising placing the composite material under sub atmospheric pressure at a temperature below the melting point of the polymer, in order to partially activate the MOF and contacting the composite material with NO gas, such that NO gas binds to both metal and water molecules present within pores of the MOF.
18. The method according to clause 17, further comprising removing excess and/or weakly bound NO gas from the composite material by flushing the composite material under a reduced pressure (such as 10-2 torr) for a few seconds (e.g. 2-30) with a dry inert gas, such as argon.
19. The method according to clauses 17 or 18, further comprising purging the composite with a dry inert gas, such as argon in order to remove a portion of the bound NO gas, such as gas not removed by flushing alone, from the composite.
20. The method according to clause 19, wherein purging comprises placing the composite material under sub atmospheric pressure at an elevated temperature for at least one hour followed by one or more cycles of flushing the composite with a dry inert gas.
21. The method according to any of clauses 17-20 for use in preparing a medical article according to any of clauses 2-16.
22. A medical article comprising a composite material obtainable by the method according to any of clauses 17-21.
23. A method of releasing a gas within a body of a subject, the method comprising inserting into the body of the subject a medical article according to any of clauses 2-16, or clause 22.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The present disclosure will now be further described with reference to the Figures that show:

FIG. 1A-1E. Extrusion of MOF-polymer catheter tubing. FIG. 1A, the activation-loading-delivery cycle for NO in MOF-74 MOFs; FIG. 1B, Images of extruded tubes, showing the control Pebax polymer only (Virgin Pebax, left) and 5% Zn-MOF in Pebax. The yellow colour is that of the MOF; FIG. 1C, X-ray Computed Tomography (XCT) image of a cross section 5% Zn-MOF Pebax tube: the bright spots are the MOF; FIG. 1D, XCT image comparing the thresholding binary output, where voxels containing MOF are rendered in blue, with the greyscale input; FIG. 1E, Equivalent spherical diameter (ESD) plot of the MOF crystallite size determined from 438,033 individual, well-dispersed MOF particles.

FIG. 2A-2D|Selected physical characterisation data of: FIG. 2A, Summary of DSC data for tubing samples; as extruded, post dehydration (D) and after NO load and release procedure (L&R); * indicates that the heat of fusion is normalised for inclusion of MOF. FIG. 2B, Young's moduli and heat of fusion of tubes. FIG. 2C, SEM micrograph of Pebax®+10% MOF with example of voidage highlighted in white circle; FIG. 20, Storage modulus (G′) and loss modulus (G″) as a function of frequency for Pebax® and Zn MOF composites.

FIG. 3A-3F|NO-release experiments. NO-release data recorded in the gas phase for; FIG. 3A, 5 wt % CPO-27-Zn Pebax tube. FIG. 3B, Virgin Pebax polymer tube. FIG. 3C, Virgin Pebax polymer tube having been subjected to a more aggressive purge protocol (four hours vacuum at 50° C.). FIG. 3D, 5 wt % Zn-MOF Pebax tube having been subjected to a more aggressive purge protocol (four hours vacuum at 50° C.) Key: Red—measured immediately after loading (“fresh”), blue—after 1 day, and green—after 6 days storage. Data collection was stopped at 20 ppb (nearing the sensitivity limit of the analyser) FIG. 3E, schematic illustration of possible adsorption sites within a MOF-polymer composite. FIG. 3F, NO-release data for 1 and 5 wt % Zn-MOF Pebax tube measured using an NO-sensitive electrode.

FIG. 4A-4C|Crystal structures of Ni-CPO-27 from in situ single-crystal X-ray diffraction studies. FIG. 4A, The structure of partially activated (353 K) MOF with ˜50% occupancy of water molecules bound to the metal (chemisorbed sites) structure of the same crystal after exposure to 2 bar of NO. Note the presence of both chemisorbed and physisorbed NO in the pores. The pink coloured molecule is the newly discovered physisorption site in this material. FIG. 4B, Fully activated (450 K) MOF with ˜6% occupancy of water on chemisorbed onto the metal sites and after loading with NO at 2 bar showing no physisorption sites for NO. FIG. 4C, Exposing fully activated then NO loaded MOF to a humid gas flow illustrates the replacement of metal-bound NO by water and the formation of a fully hydrated MOF.

FIG. 5A-5C|Toxicology of MOF-polymer tubing Effect of 15 mm lengths of Pebax5533 polymer tubing on HUVEC cell viability; FIG. 5A, MTT cell metabolism assay; FIG. 5B, LDH cell membrane integrity assay, and FIG. 5C, IL-6 release from HUVEC cells in culture (12 well plates). ****P<0.0001, ***P<0.001 Two factor ANOVA with Sidak's multiple comparison test compared to coverslip control (black bars, n=4-6).

FIG. 6A-6D. Vasodilator effect of NO release from MOF-Pebax. FIG. 6A, Schematic of the myography experiment showing the MOF-Pebax tube inserted inside a section of porcine artery. FIG. 6B, vascular relaxation of porcine coronary and radial arteries on exposure to NO-loaded 1 wt % CPO-27-Zn. FIG. 6C, vascular relaxation of porcine coronary and radial arteries on exposure to NO-loaded 5 wt % CPO-27-Zn. In FIG. 6B and FIG. 6C 15 mm tubes were inserted into porcine radial (diamond) and coronary (square) arteries. FIG. 6D, illustrates the same data as area under the curve to allow comparisons between different wt % materials and between coronary and radial arteries. *P<0.05; **P<0.01; ****P<0.0001, 2-way ANOVA with Siduks multiple comparisons test (n=6).

FIG. 7A-7B| Anti-platelet aggregation experiments. FIG. 7A, aggregometry traces generated after 2 h incubation of platelets alone (no tubing, black) or platelets in contact with Pebax® (blue), 5 wt % Zn-MOF Pebax® (purple) and NO-loaded 5 wt % Zn-MOF Pebax® (red); FIG. 7B, (Quantitative analysis of data for area under the curve (AUC) for the aggregation experiments shown in FIG. 7A; for FIG. 7A and FIG. 7B 15 mm lengths of tubing were suspended in platelet-rich plasma and platelet aggregation initiated by the thromboxane analogue, U46619 (10 mM). Aggregometry was abolished by 5% wt MOF+NO tubing, but unaffected by all but one of the other test treatments across the timecourse of the experiment. Pebax+MOF (NO-free) caused a modest but significant increase in aggregation after 1 h treatment, but at no other time point. *P<0.05; ***P<0.001, n=6.

FIG. 8|Antibacterial Efficacy. The growth control and blank tubing demonstrated no discernible difference in concentration of bacterial cell present after 24 hours incubation (p>0.05); in comparison, the viability of bacterial cells in MOF and NO-loaded MOF decreased sharply by ˜2.00 Log₁₀ CFU/mL and >4.00 Log₁₀ CFU/mL, respectively. In the instance of NO-loaded MOF no colony growth was observed during incubation. The error bars represent the standard deviation and the limit of detection for this study is indicated by the dotted horizontal line at 2.00 Log₁₀ CFU/mL.

FIG. 9|Data analysis on each trace of Tension. Tension was continuously measured via a tension transducer and Powerlab data acquisition hardware and software. The trace is described as follows: Typical myography trace. A—force induced upon activation using receptor-specific constrictors. B—reduced forced created by bradykinin additions, transient effect. C—Test material insertion, reduction in force via dilation calculated as a % relaxation from the maximal constriction minus baseline. D-ODQ addition, increase in force calculated as % recovery. A—active (agonist-induced) tension; B/A %—endothelium-dependent relaxation; C/A %—tubing-induced relaxation; and D/C %—soluble guanylate cyclase-dependent recovery.

It is to be understood that where descriptions of various embodiments use the term “comprising,” those skilled in the art would understand that in some specific instances, an embodiment can be alternatively described using language “consisting essentially of” or “consisting of.” The term “consisting” or variants thereof is to be understood to imply the inclusion of a stated element, integer or step, or group of elements, integers or steps, and the exclusion of any other element, integer or step or group of elements, integers or steps.

The term “about” herein, when qualifying a number or value, is used to refer to values that lie within ±5% of the value specified. For example, if a temperature is specified to be about 5 to about 13° C., temperatures of 4.75 to 13.65° C. are included.

Reference to physical states of matter (such as liquid or solid) refer to the matter's state at 25° C. and atmospheric pressure unless the context dictates otherwise.

An “elevated temperature” is understood to be a temperature above room temperature, so generally above 30° C., 40° C. or 50° C., for example.

Described herein is the preparation of a MOF, which is well-dispersed within a polymer material and can be extruded into a form suitable for use as, for example, a cardiovascular catheter. We describe how we prevent damage to the polymer caused by MOF-derived moisture during melt processing and show that the mechanical properties of the composite are not negatively affected by the incorporation of the MOF. We also demonstrate how partial activation of the MOF and careful control of gas-loading conditions are used to optimise the balance between gas adsorbed in the MOF compared to that adsorbed in the polymer matrix, to produce consistent gas delivery profiles. We demonstrate appropriate toxicity of the composites and show that the same composite successfully hits the three main biological targets for this type of catheter: vascular relaxation (to counter arterial vasospasm), anti-platelet activation and anti-microbial activity. Single-crystal X-ray diffraction studies on a model crystal reveal that partially activated MOFs, such as those used in the composites display different adsorption behaviour to the more commonly used fully activated materials.

It is further demonstrated how extruded MOF/polymer tubing can be prepared and how consistent delivery of nitric oxide can be achieved. It is demonstrated that prototype medical tubing that utilizes well-dispersed embedded MOF particles release NO. We discuss the practical advances required for successful extrusion of MOFs in polymer articles and, for the first time, the distribution of the MOF within the tubing is revealed in three-dimensions using X-ray Computed Tomography (XCT). The influence of the MOF on the physical properties of the composite is also assessed. Single crystal X-ray diffraction is used to understand how NO adsorption is affected by the processing conditions, and to identify a new adsorption site for NO in partially activated MOFs that is not seen in fully activated MOFs. The study is set in the context of employing MOFs as NO delivery agents in medical devices (e.g. catheters) to prevent infection, thrombosis and/or vascular/arterial spasm. To this end, the toxicology and functionality of the prototype catheter tubing is demonstrated using in vitro assays.

The present disclosure will now be further described by way of non-limiting example.

### METHODS

### Materials

All chemical reagents were of high grade purity, were obtained from common commercial sources, and were used without further purification. Pebax 5533 was obtained from Arkema's EU distributer, the National Chemical Company, formerly IMCD. Solutions used for antimicrobial testing were made up in high purity water and sterilised in an autoclave before use.

### MOF Synthesis and Characterization

Zn-MOF-74 was prepared at kilogram scale using a 100 L vessel following the room temperature, water-based procedure previously disclosed by the Morris group. Powder X-ray Diffraction (PXRD) analysis was conducted on a Panalytical Empyrean diffractometer in reflection mode operating with Cu Kα1 radiation monochromated with a curved Ge(111) crystal. Thermogravimetric analysis (TGA) was performed using a Netzsch TGA 209 instrument, in which ˜5 mg of sample was heated to 500° C. at a rate of 5° C./min in air.

### Polymer Tube Extrusion

### Melt Processing:

Production of MOF-pebax tubes was carried out using a Collins 25 mm co-rotating twin screw extruder coupled to a mono-layer tube die. An outer die with a diameter of 6.5 mm with a 4.5 mm pin was used to produce a range of tube diameters from 1.7 mm to 2.4 mm. The extrusion temperatures were from 180° C. at the feed section to 190° C. at the die. The screw speed was 100 rpm. The measured melt temperature was 194° C. The throughput was set at 0.8 kg/hr. When the extrudate exited the die it entered the sizing calibrator and cooling tank and was hauled off at a controllable constant rate, depending on the dimensions of the tube required. The tubes were then cut to size and packed in air tight bags.

### Physical Analysis of Tubing

Differential Scanning Calorimetry (DSC) was performed using a TA Instruments Discovery DSC25. Samples of extruded tubing of virgin Pebax 5533 and extruded composites of Pebax 5533 and 1&5% Zn MOF weighing approximately 10 mg were sealed in standard aluminium pans. Heating and cooling cycles were carried out under a nitrogen atmosphere at a flow rate of 50 ml/min. Samples were heated from 30 to 250° C., held for 3 mins at 250° C. to remove processing history, cooled to 30° C. and then reheated to 250° C. at a heating and cooling rate of 10 deg C./min

Dynamic Mechanical Analysis (DMA) was performed using a Triton Technologies Tritec 2000 DMA. Samples of tubes of extruded unfilled Pebax 5533 and extruded composites of Pebax 5533 and 1&5% Zn MOF approximately 2 mm in diameter were prepared in single cantilever mode. Samples were heated from −100 to 100° C. at a heating rate of 2 deg C./min at a frequency of 1 Hz and displacement of 0.05 mm

Tensile testing was performed using an Instron 5564 universal tensile tester fitted with a 2 kN load cell. Tubes of extruded unfilled Pebax 5533 and extruded composites of Pebax 5533 and 1&5% Zn MOF approximately 2 mm in diameter. The initial gripping distance was 50 mm and the samples were deformed at 50 mm/min until the elastic limit was exceeded. A minimum of five specimens were tested for each sample. Young's modulus was determined using ISO 527-2 using linear regression slope calculation

XRD analysis of tubing was conducted on a STOE STADIP X-ray diffractometer operated in Debye-Scherrer mode and employing Mo X-radiation with primary beam monochromation (MoKα₁). To enable direct comparison with as-prepared MOF material, the diffraction pattern of as-prepared MOF was also collected in this way by loading the powder into a quartz glass capillary.

SEM was used to observe the distribution of 10% Zn MOF particles in the Pebax 5533 composites using a JEOL JSM-6500F field-emission gun scanning electron microscope (FEG-SEM) under an accelerating voltage of 5 kV with a secondary electron imaging (SEI) detector. Composite samples were sputter coated with Au prior to imaging.

Rheological Analysis was performed using a TA Instruments ARG2 rotational rheometer. Pressed plaques of Pebax 5533 and extruded composites of Pebax 5533 and 1&5% Zn MOF approximately 1 mm in thickness were prepared and cut in to 25 mm diameter discs. Frequency sweeps were carried out at 0.1 to 100 Hz at a temperature of 190° C. and a strain of 0.5%.

### X-Ray Computed Tomography and Image Analysis

A 1.95 mm section of a 15 mm-long sample of the tube was XCT scanned using a 160 kVp Zeiss Versa system. The X-ray source energy was set at 80 kVp and 7 W power. A total of 3201 projections with a 2-second X-ray exposure time per projection were acquired over a full 360° sample rotation. The source-to-object and object-to-detector distance was 12.00 and 24.75 mm, respectively, which yielded 1.1 μm reconstructed voxel size with the aid of 4× light microscope detector optics. Reconstruction was carried out using filtered back projection implemented via the Reconstructor Scout-and-Scan software package (Carl Zeiss, USA).

A thresholding image processing procedure, which encompasses the classification of voxels with GVs above or below a certain threshold value, was applied to the reconstructed XCT image to extract the MOF material. Thresholding returned a binary 3D image where MOF and non-MOF voxels contained values of one and zero, respectively. The total volume of MOF within the scanned region was determined from this binary image by counting the number of MOF voxels.

A watershed segmentation procedure was then applied to the resulting binary image containing MOF particles to assign individual numbers (known as ‘labels’) to each MOF particle. Watershed segmentation involves the differentiation of features of a binary image into individual elements (a detailed description of the watershed method is available in, e.g.,^{[19}). The length-to-breadth ratio (LBR) and equivalent spherical diameter (on a volume-basis; ESD) for each particle were then produced from the segmented 3D images.

All image processing was carried out using Avizo 3D 2021.2 (Thermo Fisher Scientific, USA).

### NO Loading and Release

MOF powders and MOF-polymer tubes (xx cm long) were prepared for kinetic NO release measurements and in vitro analyses following a previously described methodology. In brief, samples were placed into glass ampoules within a Schlenk flask, the flask was connected to a Schlenk line and the samples were heated to 353 K overnight, under vacuum (1×10-4 mbar). The samples were exposed to dry NO (2 bar) for 1 hour and sealed under dry argon.

In an adaptation to the NO loading process, samples were bathed in NO for up to 4 hours. In some situations samples were purged for up to 4 hours under dynamic vacuum at 50° C. prior to sealing under argon.

Kinetic NO release measurements were recorded using a Sievers NOA 280i chemiluminescence nitric oxide analyser under flowing humid nitrogen (95% relative humidity), as described previously. Data collection was stopped at 20 ppb (nearing the sensitivity limit of the analyser), and the data were collated to generate plots of the concentration of NO (in ppm) released overtime.

### In-Vitro Toxicology, Myography and Aggregometry

Ethical approval for this work was granted by the Research Ethics Committee and the Animal Welfare and Environment Committee at the University of the Highlands and Islands (OL-ETH SHE 5325).

### HUVEC Cell Culture

Human umbilical vein endothelial cells (HUVECs) stored at −152° C. in dimethyl sulphoxide were thawed at 37° C. and resuspended before transfer to a T-75 flask in a cell culture hood with ˜12 ml of endothelial cell growth medium prior to incubation (37° C. incubator at 5% CO₂). After 24 hours, the medium was changed to remove the dimethyl sulfoxide. Medium was then changed every second day until ˜80% confluency was achieved.

### HUVEC Subculture

Medium was removed from HUVECs at ˜80% confluency and the monolayer of cells was washed twice with phosphate buffered saline (PBS; Hyclone™). Cells were trypsinised through addition of 3 ml of 0.5% trypsin/ethylenediaminetetraacetic acid (EDTA) prior to addition of medium (10 ml) and centrifugation (500 g, 3 min, room temperature). The supernatant was then aspirated and the pellet resuspended in 3 ml of PBS prior to centrifugation (500 g, 3 min, room temperature). The supernatant was aspirated and 1-2 ml of medium was added prior to pellet resuspension and cell counting. The cells were then counted using a haemocytometer. Cells were seeded at 7.5×10⁵ cells per flask (T-75) in 12 ml of growth medium; subsequent cell seeding in 12 well plates was at 1×10⁵ cells per well in 1 ml of growth medium.

### Polymer Tubing Treatment of Cells and Outcome Measures

Medium was aspirated from cells prepared as above in 12 well plates prior to washing twice with PBS, and re-immersion in 1 ml of endothelium growth medium. Meanwhile, 15 mm lengths of test tubing were lightly sprayed with ethanol and left to dry to sterilise, prior to placement in each well and covering with a glass coverslip and incubation (37° C., 5% CO₂) for 24 h, at which time cell viability assays (lactate dehydrogenase or MTT) were conducted using commercially available kits. Alternatively, the supernatant was aspirated and used to measure accumulation of the cytokine, interleukin-6 (IL-6) by ELISA. All experiments were conducted with parallel treatments that included blank polymer tubing, MOF-containing polymer tubing without NO and MOF-containing polymer tubing loaded with NO. Positive controls for the assays were H₂O₂ (500 μM) treatment for viability assays and TNF-a for IL-6 assays.

### Vasodilator Studies

### Preparation of Porcine Coronary and Radial Arteries

Pig hearts and fore trotters were obtained fresh from a local abattoir.

The left anterior descending artery and radial arteries were located and dissected out of the heart and pig trotter respectively. Arteries were immediately placed in cold, calcium free, Krebs buffer solution, cleaned of fat and adventitia and cut into ˜3-4 mm rings for mounting on a fixed wire myograph. If not being used immediately, the artery segments were stored in Krebs buffer at 4° C.

### Fixed Mount Myography

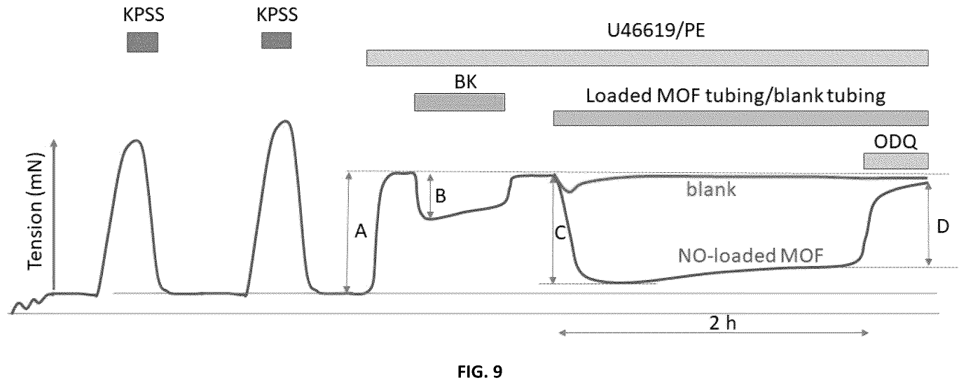

Arterial rings were mounted on the wire (for 1 h, 37° C., bubbled with 95% O₂; 5% CO₂) and normalised to a resting tension of 13-15 mN for coronary arteries and 10-13 mN for radial arteries. Following assessment of contractile viability using high potassium Krebs buffer, arteries were pre-contracted using either U46619 (1 μM; coronary arteries) or phenylephrine (3 μM; radial arteries) and endothelial integrity was tested using bradykinin (10 μM), which in the presence of endothelium causes a transient relaxation of the vessel. The NO loaded MOF material or blank control material was inserted into the lumen of the arterial ring using a bespoke insertion module and left for 2 hours prior to addition of 1H-[1,2,4]oxadiazolo[4,3-a]quinoxalin-1-one (ODQ) (0.3 mM), a selective inhibitor of soluble guanylate cyclase, to determine the extent of the role of the NO:guanylate cyclase pathway in any relaxation seen. Tension was continuously measured via a tension transducer and Powerlab data acquisition hardware and software. Data analysis on each trace was conducted as shown in FIG. 9.

### NO Electrode Measurements

Electrochemical detection of NO in solution facilitates real-time measurement of NO, with a limit of detection ˜50-100 nM. Experiments were conducted to facilitate measurement of NO in the same media used for cell culture and myography experiments in an effort to equate toxicological and physiological responses to NO exposures, albeit at the limits of detection by this method.

15 mm lengths of NO-loaded, MOF containing extruded tubing were weighed down with short lengths of stainless steel wire inserted into the lumen and placed in either 1 ml volumes of cell culture medium or 5 ml volumes of Krebs buffer at 37° C. (to match conditions of in vitro assays) in which a NO electrode was already suspended and allowed to equilibrate for up to several hours. Care was taken to ensure that the proximity of the electrode (<1 mm) was reproducible between experiments through the use of a micromanipulator for positioning. The experiment was run for 2 hours subsequent to addition of the tubing to the solution, with continuous measurement of NO throughout. On each day, the electrode was calibrated with a range of SNAP concentrations to allow subsequent conversion of electrode signal (mV) to be converted to NO amount (nmol). Data were analysed using MS Excel and Graphpad Prism (version 9.0).

### Platelet Aggregometry

### Turbidometric Platelet Aggregometry

Standard turbidometric platelet aggregation was used to assess the impact of Blank PEBAX 5533 tubing, 5 wt % Zn PEBAX 5533 MOF only tubing and NO-loaded 5 wt % Zn PEBAX 5533 MOF tubing on U46619-activated human platelets in platelet-rich plasma (PRP). Briefly, 6 healthy volunteers were recruited to the study (age 20-60 years old, x male, y female). Individuals were not fasted prior to the study but had not had non-steroidal anti-inflammatory drugs for 10 days prior to the study. A 50 ml peripheral venous blood sample was drawn from the antecubital fossa into trisodium citrate (0.38%) tubes prior to centrifugation (room temperature, 200 g, 10 min). PRP was aspirated and platelets were counted using a haemocytometer prior to normalisation to a count of 250×10⁶/ml using autologous platelet poor plasma (PPP) derived from centrifugation (1200 g, room temperature, 10 min) of the blood remaining after removal of PRP. 500 ul PRP samples were exposed to 15 mm lengths of control polymer, MOF containing polymer or NO-loaded MOF containing polymer for 5 min, 1 h or 2 h. All tubing was pre-exposed to 0.9% saline for 10 minutes prior to suspension to mimic tube flushing in clinical use. U46619 (10 μM) was added to PRP under continuous stirring and light transmission through the sample measured continuously for the subsequent 6 minutes (4-channel optical platelet aggregometer, Chronolog 460). Aggregation was quantified using the associated software (Aggrilink) and expressed as AUC over the 6 min period post-treatment. A control sample (untreated) was also tested at each of the 3 timepoints and results from samples exposed to tubing were normalised to the relevant control sample at the relevant timepoint.

### Statistics

Data were analysed using GraphPad Prism version 9.0. Statistical analysis was performed using either one-way or two-way ANOVA, as appropriate for the data. NO electrode data were first transformed using MS Excel to convert values from voltage outputs to NO amounts using contemporary standard curves for each experiment. Calculated values were subsequently analysed using Graphpad Prism (9.0).

### Antibacterial Efficacy

### Bacterial Culture Setup

Methicillin-resistant Staphylococcus aureus (MRSA) (BAA-43, American Type Culture Collection (ATCC), Manassas, USA) used in this study was sub-cultured into 10 mL of tryptone soya broth and incubated overnight at 37° C. The number of colony forming units (CFU) per mL of bacterial suspension was adjusted to 0.5 McFarland (approx. 10⁸ CFU/mL) and further diluted 1:10 to a working culture of 107 CFU/mL into 1:500 strength tryptone soya broth (Thermo Scientific, UK).

### Direct Contact Method

The tubing samples, a total length of 10 cm was used for each measurement were placed into 15 mL falcon tubes and inoculated intraluminally with 100 μL of the bacterial suspension; the samples was then incubated for 24 hours at 37° C. The samples were then submerged into 1 mL of Dey Engley neutralising solution (Millipore, UK), and the intraluminal surfaces flushed with the solution to rinse the planktonic and reversibly attached bacteria from the tubing surface. The tubing was then aseptically cut into smaller sections (1-1.5 cm) using sterile scissors to ensure complete coverage of the Dey Engley neutralising solution before being sonicated for 30 minutes.

A sample of the inoculum was taken initially, at T=0, and enumerated to confirm the inoculum bacterial cell density. As a growth control, the bacterial suspension used to inoculate the tubing samples was also sampled at 24 hours after incubation at 37° C. and subjected to identical experimental conditions and processed in parallel with the tubing samples.

The resultant solutions were enumerated to determine the bacterial cell density (Log₁₀ CFU/mL) by serial diluting in phosphate buffer solution (PBS) (EO Labs, Bonnybridge, UK), plated onto tryptone soya agar (TSA, EO Labs) and incubated overnight at 37° C. before counts were made.

### Experimental Design

In this study, a total of 9 tubing samples were tested across three discreet separate experimental runs. The bacterial cell density was measured as Log₁₀ CFU/mL before statistical calculations were performed using these values. Any statistical difference between the growth control and the tubing variants was determined using one way analysis of variance. The variation for each tubing type, blank, MOF and MOF+NO (1 hr) was given as a standard deviation of the mean and statistical significance of data sets was evaluated with GraphPad PRISM® (ver. 8.04) using one-way ANOVA.

### Example 1: Preparation and Characterisation of Extruded MOF-Pebax Tubes

### a) MOF Selection and Choice of Polymer

The MOFs CPO-27-Zn and CPO-27-Ni (also known as Zn-MOF-74 and Ni-MOF-74) were selected for this study. CPO-27 materials possess a honeycomb-like structure composed of metal ions connected by 2,5-dihydroxyterephthalic acid¹⁶. CPO-27/MOF-74 is an important MOF structure that can be prepared with many different metal components and that is studied for a wide range of applications. It has been shown previously that this material is very effective at adsorbing NO¹, as well as other gases. The gas binds to coordinatively unsaturated metal sites created in the structure upon activation. Importantly, adsorbed NO can be released from the MOF when the material is exposed to moisture; water enters the internal pore structure and displaces the bound gas. An important feature of this MOF is therefore its hydrophilicity. CPO-27-Zn can also be prepared at large scale without using any toxic solvents. This was an important consideration for a study such as this that requires several hundred grams of material, and it is also important to consider for future scale-up and industrial adoption. As a targeted application in this case is a medical device, it is important to avoid the use of any undesirable metals, solvents or additives that may transfer to the end-product and impart cytotoxicity. For this study the CPO-27-Zn MOF material was prepared at multi-Kg scale using a water-based batch process¹⁷.

The main component of the composite will be the polymer matrix. For this we chose a commercially available material, Pebax® 5533 that is already used in the manufacture of minimally invasive medical devices. Pebax® 5533 is a thermoplastic co-block elastomer made of flexible polyether and rigid polyamide blocks. Since the trigger mechanism for release of NO from the MOF is moisture, an important property of the chosen polymer is its hydrophilicity, which can be quantified by the water adsorption value of the polymer. For Pebax® 5533 this is 1.2% (measured over a 24 hr period in water at 23° C., as per ISO62)—this is ideal for our purposes as this figure is relatively low, which allows control over the ingress of water into the composite, which then controls the rate of NO release.

### b) Tube Extrusion

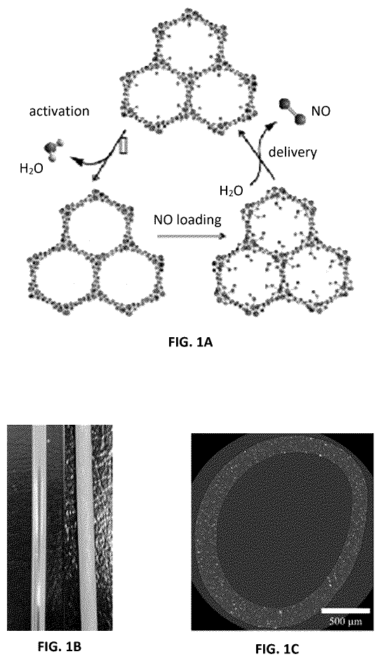

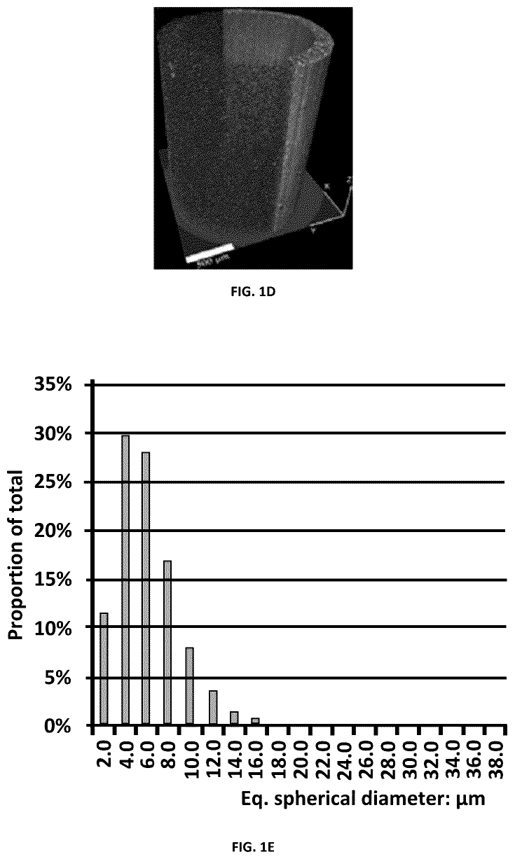

The major challenge of extruding the composite tubing is the hydrophilic nature of both MOF and polymer materials. The thermal pathway required for successful extrusion means that if there is any water within the MOF prior to the process, it can be desorbed during the extrusion, volatilising under the high temperature and pressure generated, which leads to poor quality tubing. In addition, the MOF is hygroscopic enough that re-adsorption of water needs to be avoided even if the starting MOF is fully dehydrated. If moisture levels are not sufficiently reduced during the entire process then the composite material properties can be severely affected, ultimately leading to degradation of the polymer molecular structure. The drying protocols required are therefore significantly more complex than when extruding the polymer alone, exacerbated by the fine powder form of the MOF and the need to prevent readsorption of water. This required the dried material to be kept under a dry nitrogen gas atmosphere prior to it being fed directly into the extruder. Without taking care to control moisture, extrusion was unsuccessful or produced poor quality tubes, and only through using this protocol, we successfully prepared MOF-Pebax® tubing (FIG. 1B).

### c) Physical Properties of the Composite

### Crystallinity and Dispersion of MOF in Composite

X-ray diffraction patterns of MOF-Pebax® tubes were determined along with data collected for as-prepared CPO-27-Zn and the Pebax® polymer. The pattern from MOF-containing tube shows a broad and high background contributed by the polymer, on top of which are observed peaks characteristic of the MOF. These data suggest that the MOF has long-range structural integrity following the extrusion procedure.

X-ray Computed Tomography (XCT) was employed to assess the distribution of MOF within the tubing. This is an imaging technique in which radiographs of an object are acquired from different positions following a rotational trajectory. XCT typically produces a 3D greyscale image of the scanned object, where the different grey values (GVs) contained in each voxel (3D pixel) denote local variations in X-ray attenuation.

A typical cross-section slice and a rendered view of the reconstructed volume, collected from a 1.95 mm section of a 15 mm length of tubing containing 5 wt % CPO-27-Zn, are shown in FIG. 1C-1D. The brighter spots indicate MOF particle locations, due to their higher X-ray attenuation compared with the polymer matrix. The results indicate that the MOF particles are well distributed throughout the entire thickness of the tube wall, and along its length.

FIG. 1D compares the thresholding binary output with the greyscale input; voxels determined to contain MOF in half the of the measured volume are rendered in blue. From this binary image, the total volume of MOF measured amounted to 0.065 mm³, leading to a mean MOF concentration of approximately 3.26% by volume within the scanned length. A total of 438,033 individual MOF particles were identified within the 1.95 mm length of tube scanned. The length-to-breadth ratios (LBR) of the crystallites vary between 1 and 2. This is consistent with the shape of the MOF particles in the as-made powder and the equivalent spherical diameter (ESD on a volume-basis) quantification results (presented in FIG. 1E as percentages of the total number of MOF particles detected in the scan). Over half of the MOF particles have an ESD between 4 and 6 μm.

### d) Polymer Crystallisation Process

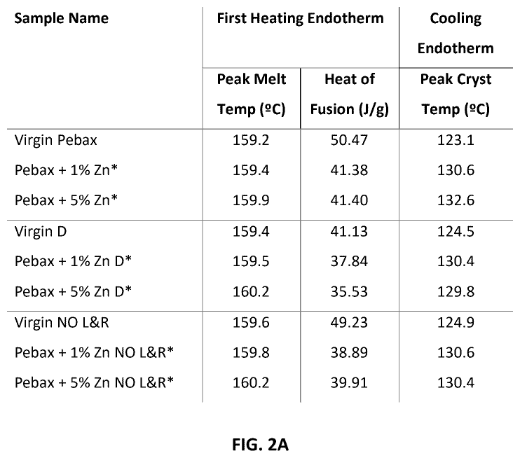

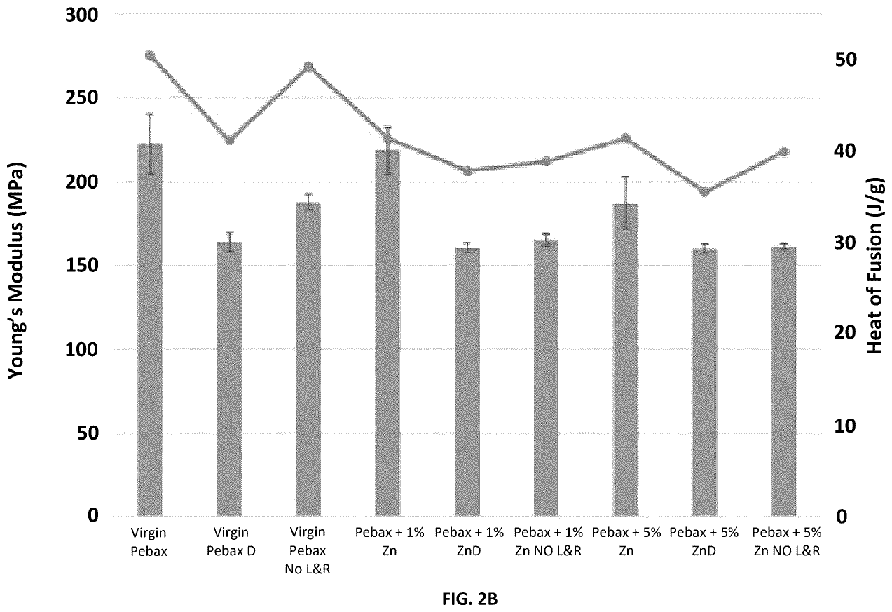

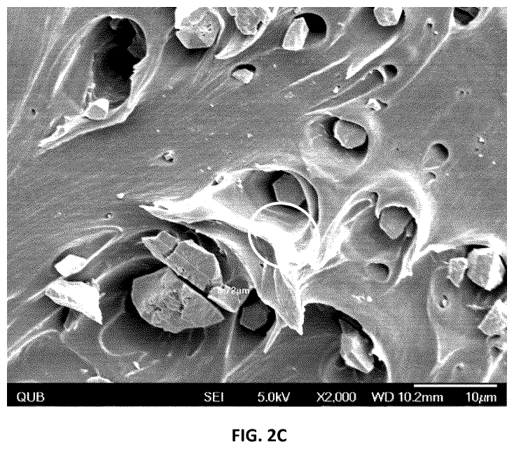

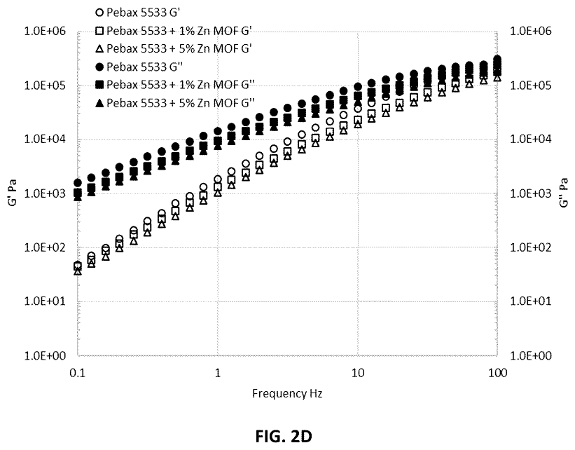

A summary chart of the peak melting temperature and heat of fusion from the first melting endotherm and the peak crystallisation temperature is shown in FIG. 2A. Melt endotherms from the first heating process and subsequent cooling endotherms of the tubes were determined. It is evident that the addition of MOF particles at 1 and 5% does not affect the peak melting temperature of the hard segment of the copolymer and therefore its overall crystalline size distribution. However, the heat of fusion and the amount of crystalline content is significantly lowered by addition of MOF. This suggests that the polymer molecular chain mobility and subsequent ability to crystallise from the melt has been reduced by the presence of the MOF particles. Hindered molecular chain mobility is further confirmed by the significant increase in peak crystallisation temperature on addition of the MOF.

It is also noted that the post processing steps of dehydration and NO loading and release (discussed below) also significantly reduce heat of fusion. Dehydration at 80° C. results in a reduction in heat of fusion of the primary melting process alongside a growth of a secondary shoulder at a lower temperature, indicating the formation of a smaller crystalline form. This secondary shoulder is retained during the NO load release process, though at a lowered temperature. This loss in crystallinity and formation of a secondary shoulder would indicate that post processing affects the stability of the polymer's crystalline structure which in turn will affect product performance characteristics such as stiffness. Caution is required to strike a balance between altering the polymer properties alongside the post processing requirements.

### e) Glass Transition Process

There was no discernible effect on the amorphous glass transition process of Pebax® with the addition of the MOF particles at 1 or 5%. This would suggest there is little disruption of the molecular free volume on addition of the MOF particles.

### f) Mechanical Properties

The effect of inclusion of MOF particles and the post processing steps on the Young's modulus of the tubes are shown in FIG. 2B. The break strength of the tubes was determined. It is clear that addition of 1% MOF has negligible effect on the modulus and break strength of the tubes, but at the higher addition of 5% MOF there is a marked reduction in both modulus and break strength. This would suggest that the MOF particles are not intimately bound to the polymer matrix, creating a boundary void and reducing the strength of the composite. A scanning electron microscopy image in FIG. 2C shows a lack of bonding between the MOF particles and the polymer matrix to support this.

The Young's moduli of the tubes is shown to decrease upon dehydration, and in the case of the virgin tubes, recover partially with the NO load and release process. This change in moduli trend corresponds with the underlying change in heat of fusion caused by post-processing which would suggest that restructuring of the crystalline content is a major contributory factor to the ultimate mechanical performance of the tubes.

### g) Rheological Behaviour

The storage modulus (G′) and loss modulus (G″) of the MOF-Pebax® composites as a function of frequency are shown in FIG. 2D. Typical melt behaviour is observed with viscous response (G″) to frequency being dominant across the range of frequencies. Small stepwise decreases in both G′ and G″ on addition of MOF suggest a lubrication effect from the particles on polymer melt flow, especially at higher frequencies. Linearity in the terminal region at the lowest frequencies, where visco-elastic properties are most sensitive to molecular architecture interactions, suggests no restriction on chain mobility due to inclusion of the MOF particles.

A Han plot of G′ versus G″ was determined. The plot again shows viscous dominant behaviour with linearity at the lower moduli section of the plot showing no restrictive interaction on chain mobility.

The small changes in G′ and G″ on inclusion of 1 or 5% MOF would suggest little to no detrimental effect on the processability of the polymer, and confirm that moisture uptake is the main contributory factor to the processing challenges. The observed reductions in moduli, which are up to 30%, are in part due to MOF inclusion and in part to post-processing. However, as these moduli reductions are in an otherwise flexible polymer, this would suggest no appreciable change to mechanical haptics of the tube or any medical device developed from the tube, which is particularly pertinent within clinical settings.

### Example 2: NO Release from MOF-Pebax® Tubing

To render the internal surface area of the MOF accessible and to remove coordinated water to generate binding sites for NO, the MOF must be subjected to an activation process. Typically, this is achieved by heating the MOF under vacuum. For CPO-27-Zn, temperatures in excess of 150° C. are generally required in order to achieve full activation. However, such high temperatures are incompatible with many polymers, including Pebax®; heating tubing to such high temperatures would result in the polymer melting, which would be incompatible with the processing. Therefore in a MOF composite the activation temperature will necessarily be much lower than for pure MOF—in the case of MOF-Pebax® a temperature of 80° C. (353 K) leads to a reduction in, but still surprisingly significant, NO loading capacity. The MOF-polymer composites were therefore loaded with NO according to the protocol in the Materials and Methods section.

Two techniques were employed to characterise the release of NO from processed tubing. First, a gas-phase Nitric Oxide Analyser (NOA) was utilised to characterise, screen and compare formulations and processing conditions. This technique uses humid (95% Relative Humidity) nitrogen flowing over the sample to trigger the release of NO. The NO is subsequently carried to the analyser by the flowing nitrogen and is detected in the gas phase. Second, lead formulations were characterised using an NO electrode, which offers the opportunity to measure NO release in liquid media that is more appropriate for the targeted application environment and release trigger. Results from the NOA will be discussed first.

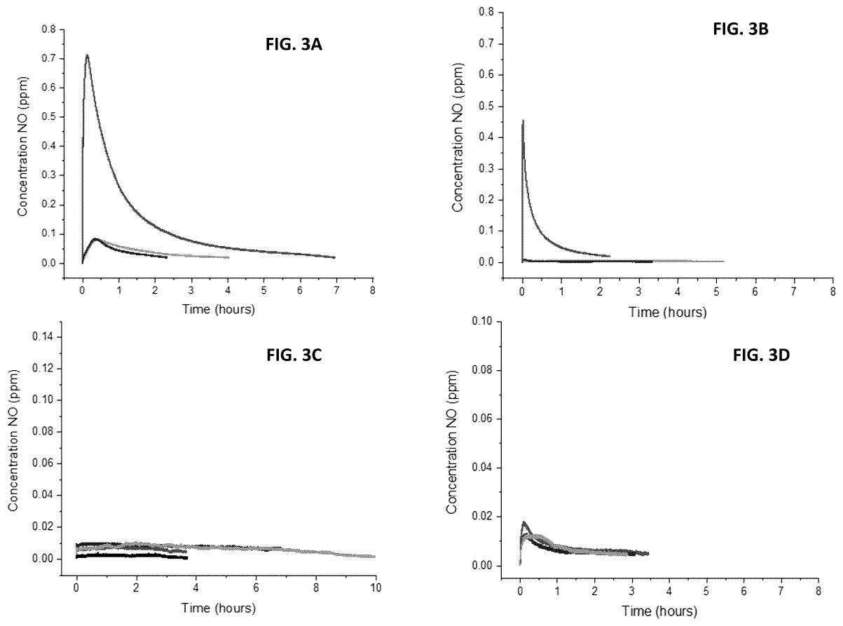

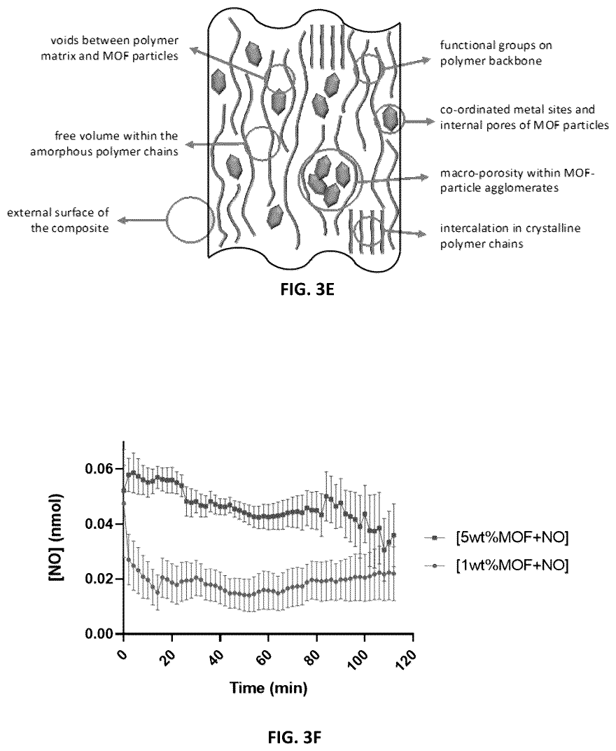

FIG. 3A shows the NO delivery profile of a tube containing 5% CPO-27-Zn measured on the NOA. A freshly loaded tube shows a large spike in NO that reduces over time. After storing the NO-loaded tube under dry argon (a process referred to as aging), this spike is much reduced. Release profiles for aged samples appear to converge to a lower value after six days. A similar effect is observed for a tube loaded with 1% CPO-27-Zn though the amount of NO released is much lower, as would be expected from having a lower MOF content. An inconsistent release of NO with respect to storage time would be unacceptable for any application and it was therefore necessary to investigate this issue.

FIG. 3B shows the NO delivery profile of a tube made only from Pebax® (i.e. without MOF) after being subjected to the same NO loading process employed above. It reveals the polymer itself has the ability to adsorb and release NO. In a similar manner to the MOF-containing tubes, the freshly loaded Pebax® tube shows a spike of NO that subsequently dies away. On leaving the samples to age, this spike is much reduced and after storage for six days is essentially absent. It was therefore postulated that the aging effect observed for MOF-loaded tubes may be caused by a component of NO that is less strongly adsorbed within the polymer rather than that more strongly bound within the MOF.

To rationalise the behaviour of these MOF-loaded tubes it is necessary to consider all potential adsorption sites within the composite. These are illustrated in FIG. 3E. The mechanism of adsorption within the MOF crystals is chemisorption of the NO on the open metal sites (as has been shown by single-crystal X-ray diffraction¹⁸) whereas there are likely to be various physisorption sites within the polymer and composite, as illustrated (there are no chemical binding sites for NO on the polymer backbone). As only physisorbed sites exist in the polymer itself, it was proposed that physisorbed NO may be the cause of the aging effect. Physisorbed gas should be easier to remove from the composite than the chemisorbed NO within the MOF. Therefore, to test this hypothesis, a more aggressive purging regime was developed in which the NO loaded polymer tubing (without MOF) and MOF-loaded polymer tubing were subjected to four hours under vacuum at an elevated temperature of 50° C. (rather than only brief vacuum removal of excess NO at ambient temperature). The resulting NO release profiles (as measured by NOA) for Virgin Pebax polymer without MOF (FIG. 3C) demonstrate zero release at all time points, consistent with any NO physisorbed into the polymer being removed by the more aggressive purge. The effect of applying the more stringent purging regime to MOF-containing tubes is demonstrated in FIG. 3D for tubes containing 5 wt % MOF. By comparing FIG. 3A and FIG. 3D it is apparent that the purging approach has indeed alleviated the issue of aging, allowing the true contribution to the release profile from the MOF can be observed. The results suggest that the major complication in obtaining stable and consistent NO delivery stems from the necessary porous nature and adsorption capacity of the Pebax® polymer. Our results indicate that the aging effect observed in the NO release profiles of both MOF-loaded polymer and polymer without MOF is driven by a physisorbed component, which is slowly lost into the headspace on storage.

In a further development of the NO loading protocol, we have found that by bathing the samples in NO for longer (e.g. four hours), the quantity of NO released from the composites can be increased and the timescale of release can also be elongated. This demonstrates that the dose and release profile of NO can be further refined by altering the processing conditions, as well as by altering the formulation itself. This provides yet further opportunity to achieve the required profile for the targeted biological response.

While the above approach to analysing NO release is valid when characterising and comparing the materials, it is less relevant to the final application and to the environment in which the tubing will be required to function. To reflect the application environment more appropriately, the samples were fully immersed in media and the NO release was quantified using NO-specific electrodes (see Materials and Methods). Furthermore, it should be noted that it is normal practice in the catheter laboratory of a hospital, for example in the preparation before a percutaneous intervention, to flush the required devices with saline. The whole preparation time can last approximately 10 minutes, during which time the devices are exposed to ambient conditions. Since NO release is triggered by moisture, we wished to closely mimic this operation in our analysis protocols to provide a more realistic model of a real-life situation. Therefore, tube samples were exposed to 0.9% saline solution and left on the bench for 10 minutes prior to conducting the analysis. This protocol was also followed in the preparation of samples for the biological assessments discussed below.

FIG. 3F shows the NO release profiles of tubing containing 1 and 5% MOF, as measured in this alternative approach. It is evident that the tubing containing 5% MOF releases larger quantities of NO compared to that containing only 1%, as would be expected. Also, the amount of NO measured from the samples is much lower than that measured in the gas phase analysis above. This may be a result of the saline flush, but it may also be due to the differences between the techniques. For example, the electrode records NO being released from a very localised area of substrate.

Notwithstanding the differences between the two methodologies employed, the data from each technique indicate NO release can be achieved at what is regarded to be within the therapeutic range of about pmol to nmol concentrations. Furthermore, the duration of release is sustained over a period of up to a few hours under the conditions employed. Such a duration would be ideally suited to application in devices (e.g. catheters) employed in percutaneous interventions (e.g. percutaneous coronary intervention), which typically last less than one hour, and a maximum of around five hours.

### Example 3: Single-Crystal X-Ray Diffraction Experiments

Typically, adsorption experiments using MOFs rely on an activation process to remove any residual solvent (or other guest molecules). This leaves the pore space in the material empty and accessible to guest molecules that are adsorbed. Activation of MOFs normally involves removing all the guest molecules; however, in the case of Zn- and Ni-CPO-27 the temperature required for this is well above the temperature that the Pebax polymer can tolerate and the activation temperature is limited to 80° C. (353 K). An important question is therefore—how does this lower activation temperature affect the adsorption of NO? To probe this single-crystal X-ray diffraction was carried out on samples of Ni-CPO-27 at the Diamond Light Source. A selected crystal of Ni-CPO-27 was mounted inside an environmental cell as described previously and activated at 353 K for 9 hours under vacuum. At this temperature the metal bound occupancy of water plateaued at 48%. This is less well activated than in our previous work, but the conditions used better replicate that used in the activation of the MOF@polymer composites. The sample was then cooled to 300 K upon which the water occupancy increased slightly to 53%; such increases have been observed in our previous work. This suggests that the activation protocol used in the membranes is insufficient to fully activate the MOF and that approximately only one in every two metals are undercoordinated under these activation conditions.

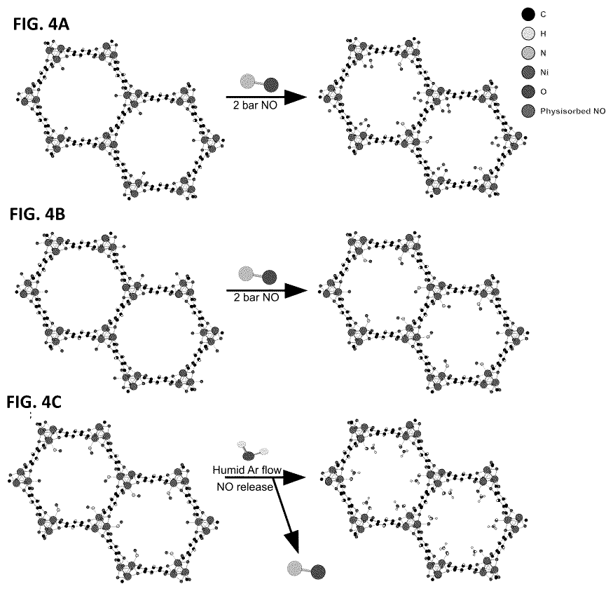

NO was then introduced to the system at different pressures from 0.01 bar up to 2 bar (FIG. 4A-FIG. 4C). It was possible to model the loaded system with an NO molecule bound to the metal site and a physisorbed NO present between the metal sites running down the length of the pores. Both sites increased in occupancy with increasing pressure following a type I isotherm, plateauing above 0.4 bar. First, the NO binds to the remaining open metal sites, then it binds into the physisorbed site. NO is only observed to bind into the physisorbed site when there is water present at the metal site. In our previous work when we loaded Ni-CPO-27 with NO after a full activation (<10% water occupancy) this physisorbed site was not observed.^{2,3}. The additional physisorbed site for NO in partially activated CPO-27 is surprising, and indicates that the constraints placed on the thermal processing of the MOF by the polymer are not as disadvantageous as one might have expected considering the information previously known about NO adsorption in this MOF.

To replicate the purge process undertaken after loading of the MOF/polymer composites we initially subjected the crystal to three rounds of vacuum/argon cycling. This caused no change to the chemisorbed occupancy but caused the physisorbed occupancy to decrease by 17%. This shows that the purge treatments on the MOF/polymer tubing probably have only a small effect on the NO loading inside the MOF, confirming that the decrease in NO delivery after such treatment is primarily due to removal of NO that has been physically adsorbed into the polymer, supporting the hypothesis put forward in the previous section.

To further understand the release of NO from NO@Ni-CPO-27, we again used in situ scXRD at the Diamond Light Source synchrotron to uncover how water interacts with NO within Ni-CPO-27. This involved the use of a flow cell which allowed us to first activate and load a sample with NO and then flow wet argon through the cell, mimicking what happens in our devices.

A single crystal of Ni-CPO-27 was activated at 450 K for 3 hours under dynamic vacuum. The sample was then loaded with NO by exposing it to a 2 bar NO atmosphere producing a system with metal bound NO and water at 81% and 19% occupancy respectively. Having removed almost all the water during activation, no significant physisorbed NO could be successfully modelled.

The system was then flushed three times with vacuum/argon cycles, causing a reduction in NO occupancy to 76%. Then 95% relative humidity (R.H.) argon was flowed across the crystal. This caused a rapid exchange of NO with water with 96% of the NO being removed in 3 minutes and full removal within 10 minutes. The same crystal was then reactivated at 450 K for 3 hours under dynamic vacuum, loaded with 2 bar NO and the system flushed with three vacuum/argon cycles. The NO occupancy was 47%. 11% R.H. argon was then flowed across the crystal, again causing NO to replace water. However, this time replacement was slower with only 70% of the NO being removed in 3 minutes.

### Example 4: In Vitro Toxicological, Vasodilatory, Anti-Thrombotic and Anti-Bacterial Assessment of Tubing

### a) Toxicology

Biocompatibility is a prerequisite for any potential new material being developed for application in healthcare. While physiologically generated NO is generally considered to be non-toxic, higher concentrations of NO generated under pathophysiological conditions or from exogenous sources has the potential to inhibit cell respiration and induce toxicity. Toxicity studies were therefore required to not only assess the viability of the cell culture model in the face of material contact with endothelial cells, but also to determine whether NO release from materials was sufficiently high to induce toxicity.

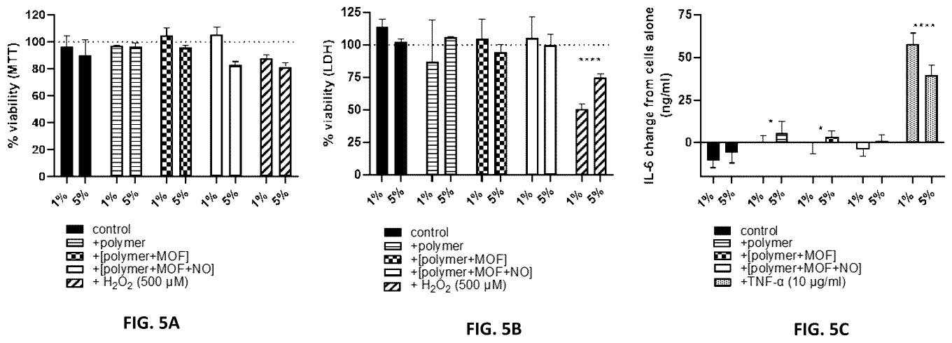

In this study, toxicity was assessed towards Human Umbilical Vein Endothelial Cells (HUVEC) using the well-established MTT and LDH assays. HUVEC cells were selected as a close mimic of arterial endothelial cells, with which the envisaged end-product devices would come into close contact. Results of the analyses are summarised in FIGS. 5A and 5B. The data from each assay indicate that none of the materials tested (virgin Pebax® polymer, [polymer+MOF] or [polymer+MOF+NO] at the 1 wt % or 5 wt % MOF loading concentrations) had any additional impact on cell viability over and above the coverslip control after a 24 hour exposure period. Overall, these data suggest that any NO generated from the MOF-loaded materials is below the threshold for induction of toxicity and the presence of MOF in the polymer has no impact on toxicity.

Inflammatory response was assessed using the IL-6 ELISA assay, also against HUVEC cells. The data are presented in FIG. 5C. In contrast to the results of the MTT and LDH assays, the impact of polymer tubing on inflammation is a little more complex, not least because the coverslip control induced a small, unexpected reduction in IL-6 production in endothelial cells which was reversed by polymer and [polymer+MOF] tubing. The cause of the modest reduction in inflammation induced by the coverslip is unknown; dogma would suggest that any nutrient and oxygen deprivation on account of the coverslip would induce, rather than inhibit inflammation, suggesting that oxygen insufficiency is not responsible. That blank polymer and [polymer+MOF] tubing reverse this anti-inflammatory impact of the coverslip perhaps suggests a mild pro-inflammatory effect of these materials. What is important to note is that the NO-loaded equivalent does not show a significant pro-inflammatory effect compared to the coverslip control, suggesting a mild anti-inflammatory effect of NO under these circumstances, in keeping with its reputation as an anti-inflammatory agent when generated at physiological concentrations; the opposite is true for NO generated at higher pathophysiological concentrations, where it is considered to be pro-inflammatory. Taken together, therefore, our data suggest that NO generated from tubing under the conditions of this experiment is at a concentration capable of anti- rather than pro-inflammatory activity. Subsequent physiological experiments were therefore conducted in the comfort of the knowledge that none of the materials tested under cell culture conditions had a substantial impact on endothelial cell viability or inflammatory activation, despite their direct contact with a proportion of the cells.

### b) Vascular Relaxation

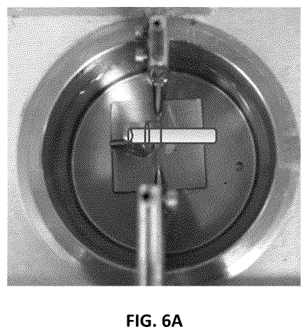

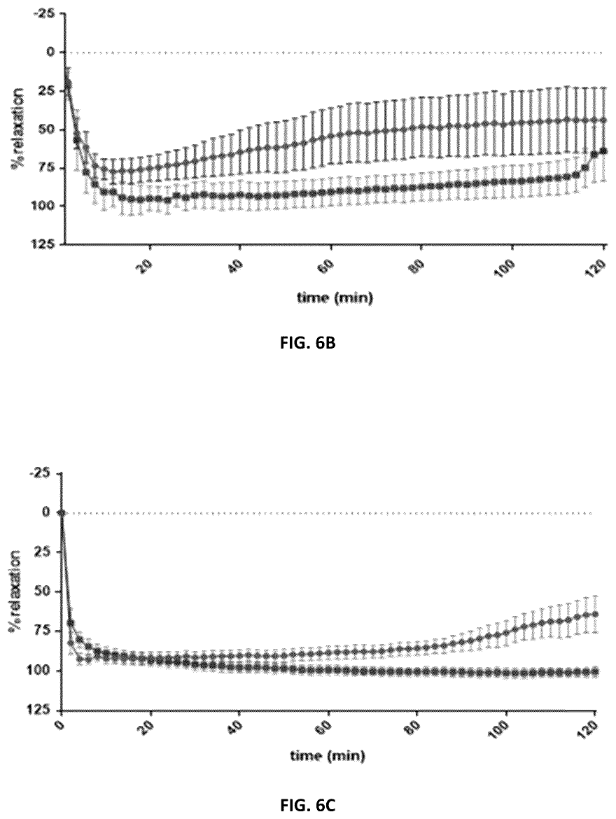

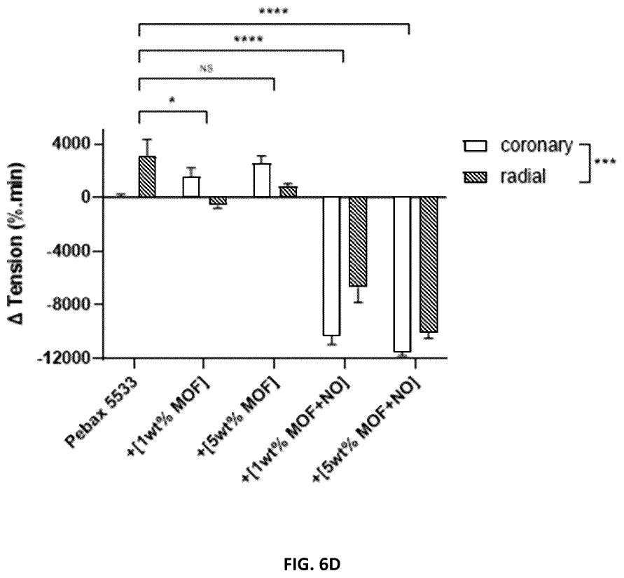

The insertion of catheters into particularly narrow and/or muscular arteries can induce spasm that is both painful for the patient and problematic for the clinician. The release of NO from the surface of the deployed devices has the potential to overcome this, eliciting a localised vasodilatory response. The vasodilatory action of the tubing could have been assessed by bringing the test articles into close proximity with arterial rings supported on the standard wire mounts of the myograph. However, it was considered that inserting the tubes into the lumen of the arterial segment would be a more realistic approach—one that would more closely mimic the intended use of the materials. Arterial segments are extremely sensitive to touch, creating a shift in baseline tension. This makes it extremely difficult to perform the insertion effectively (considering also the small diameters of the artery and tubing). Therefore, a new device was designed and manufactured specifically for this purpose. The device allowed the arterial ring to be opened vertically in addition to the horizontal stretching applied by the myograph wires, and thus permitted the insertion of tubing up to 2 mm in diameter into the arterial ring without undue perturbation of the artery or generation of background “noise” in the myograph reading (FIG. 6A). Inevitably, the additional manipulation of the arterial rings compared to standard myography led to some endothelial damage, as confirmed by sub-maximal relaxation in response to the endothelium-dependent vasodilator, bradykinin. However, this was considered to be an appropriate model for the clinical situation in which sheath, cannula and guide wire insertion used in the real process similarly induce endothelial damage. In order to closely model the real-life use of the tubing (as outlined above), samples were exposed to 0.9% saline solution and left on the bench for 10 minutes prior to conducting the analysis.

FIGS. 6B-6D summarise the results obtained from these analyses. Insertion of 15 mm lengths of [MOF+NO]-loaded Pebax® polymer tubing into the lumen of precontracted porcine coronary and radial rings elicited substantial and prolonged relaxation in both types of vessel (FIG. 6B and FIG. 6C). The relaxation was significantly larger with 5 wt % CPO-27-Zn than 1 wt % and in coronary arterial rings compared to radial artery rings. Addition of the sGC inhibitor, ODQ to the myography bath after 2 hours' exposure to the tubing induced recovery 69-98% of the initial tension, confirming that the vasolidatory effect was primarily by activation of the NO:sGC pathway. The control virgin Pebax polymer and unloaded [Pebax+MOF] tubing either failed to cause relaxation or acted to induce further contraction—this is similar to catheter-induced vasospasm that is a common complication in the medical interventions. The differential sensitivity to NO between coronary and radial arteries is of unknown origin but could be due to differences in availability of the downstream target (smooth muscle soluble guanylate cyclase) or abundance of phosphodiesterases to clear functional cGMP. Taken together, however, these experiments confirmed that profound relaxation was induced by NO delivered from loaded materials and the effect was maintained throughout a 2 hour exposure, particularly with the 5 wt % MOF material.

### c) Platelet Aggregation

Catheter Related Thrombosis is another complication often encountered during prolonged catheterisation. Thrombi can also form, however, during shorter-term catheter use, for example during minimally invasive techniques such as percutaneous coronary intervention. It develops as a result of puncture injury to the vascular wall, which promotes blood hypercoagulability through endothelial cell damage and altered haemodynamics. Platelet activation is a further possible causal feature of thrombosis which is induced by exposure of blood to foreign materials.

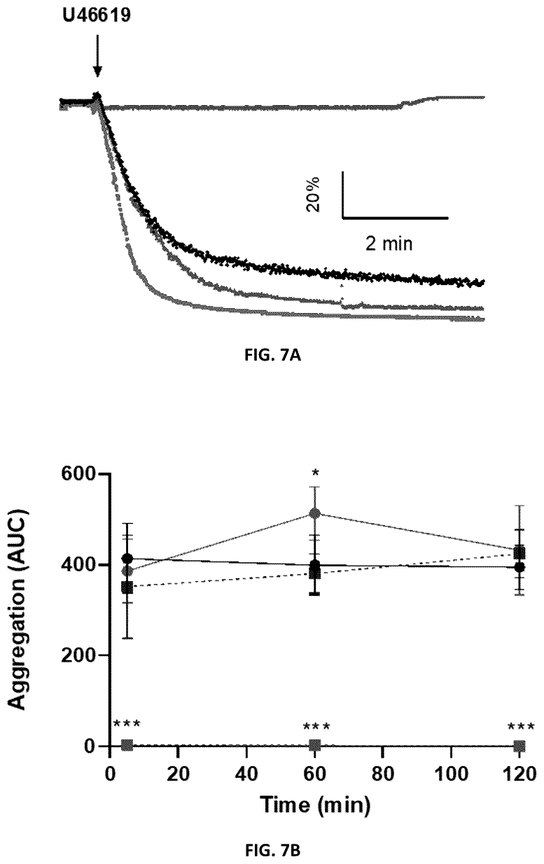

Optical platelet aggregometry is a widely used method to measure platelet function in research and medicine. This technique determines the percentage platelet aggregation in platelet-rich plasma (PRP) by measuring an increase in light transmission in response to the addition of a platelet agonist to the suspension. Platelets were incubated with and without 15 mm lengths of pristine Pebax® tubing, tubing containing 5 wt % MOF and tubing containing 5 wt % MOF that had been NO loaded. In a similar approach to that adopted for other experiments, tube samples were exposed to 0.9% saline solution and left on the bench for 10 minutes prior to suspension in PRP to mimic the saline flush and preparation time typical of a catheterisation procedure. All incubations were conducted for 5 min, 1 h or 2 h, at which point the platelet agonist U46619 was injected into the PRP samples to stimulate aggregation. FIG. 7A shows typical aggregation traces after 2 h incubation with test tubing, clearly showing the abolition of aggregation in the NO-loaded tubing. Data from 6 replicates of the experiment are illustrated in FIG. 7B, with data expressed as area under the curve for responses to U46619. NO-loaded tubing abolished aggregation at all timepoints, whereas Pebax® only tubing, and that incorporating 5 wt % Zn-MOF without NO largely had no effect on aggregation. The exception was 1 h incubation with 5 wt % Zn-MOF without NO, which showed a small but significant increase in aggregation. The extent of the effect of NO-releasing tubing is impressive, particularly when compared to the durability of U46619-induced platelet responses observed throughout the same timecourse in the presence of polymer only and unloaded [polymer+MOF] tubing.

In summary, NO-loaded, 1 and 5 wt % MOF-containing polymers have the capability of inducing powerful vasodilator and anti-platelet effects without inducing toxicity or inflammation. The localised release of NO from the surface of foreign materials that might otherwise stimulate local detrimental vasospasm and thrombosis is a distinct advantage over delivery of systemic drugs to achieve these outcomes; the free radical nature of NO ensures a very short duration of effect, equating to local rather than systemic impacts.

### Example 5: Antibacterial Assessment: Bacterial Cell Density

The costs associated with catheter-related bloodstream infections are particularly high and contribute to more than 25% bloodstream infections. It has been reported antimicrobial resistance is associated with increased costs, length of admissions and mortality rates. This study utilises MRSA as the bacterial contamination due to its inherent role within hospital acquired infections; as well as being amongst the ESKAPE pathogens, a group of the most prevalent antimicrobial organisms in healthcare. S. aureus is known to colonise the skin flora of hospitalised patients, and most notably at the insertion site for catheters; ultimately increasing the risks of bloodstream infections by these microbes. Despite many preventative therapies existing to minimise infection including catheter cleaning and early removal from patients there remains a high rate of bloodstream infections.

The direct contact method used here was adapted from the Standard method for plastic surfaces (BS ISO 22196:2011 “Measurement of antibacterial activity on plastics and other non-porous surfaces”) to make it applicable to antimicrobial tubing and more relevant for catheters. The standard method spreads 100-400 μL of inoculum (10⁶ CFU/mL Staphylococcus aureus in 1/500 dilution of nutrient broth) onto flat sample plaques, covered by an inert plastic film to define a contact area of 4-16 cm². This is equivalent to 25 μL/cm² of sample surface. Whereas the adapted method uses tubing of inside diameter 1.2 mm, which when filled with inoculum gives a very similar loading of 30 μL/cm² of tubing wall, albeit in a different geometry without the necessity of an inert cover film. The 100 μL of inoculum solution used for each measurement was thus in contact with 3.33 cm² of tubing wall.

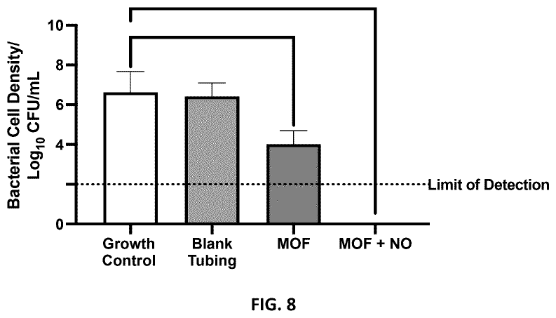

The results of the study, employing tubes containing 5 wt % MOF, are summarised in FIG. 8. The growth control demonstrated a sustained bacterial growth and viability of 6.63±1.04 Log₁₀ CFU/mL after 24 hours exposure. In comparison, a significant decrease in bacterial cell viability was observed across all tubing types (p<0.001); with complete inactivation being shown for MOF+NO, equivalent to >4.63 Log₁₀ CFU/mL reduction. Blank tubing exhibits a log reduction of <0.50 Log₁₀ CFU/mL; whilst the MOF tubing without NO achieves some antibacterial activity<2.00 Log₁₀ CFU/mL.

The results suggest that the metal organic framework alone exhibits an antimicrobial effect on MRSA in the absence of the nitric oxide. It is difficult to determine the antibacterial function present; however, it may be a result of the metal ion component (Zn in this case), or it may derive from a physical effect. For example, porosity at the surface could prevent the bacteria from being released back into the surrounding solution during processing. In contrast, NO is a known broad spectrum antimicrobial agent capable of causing cell damage through oxidative and nitrosative reactions such as DNA damage through reactive nitrogen species or nitrosation of membrane proteins. NO has known antimicrobial properties towards antibiotic resistant strains including MRSA, and it is a prime alternative treatment since there is limited evidence to support bacteria developing resistance to exogenous exposure. In this instance, by incorporating NO into MOF-containing tubing, the materials were capable of inactivating >4 Log₁₀; supporting the use of NO as an antimicrobial therapy.

It is evident that by incorporating NO within the polymer tubing, complete inactivation (below limit of detection) of a bacterial suspension is achievable. The short duration of effect is sufficient enough to instigate an efficacious response and prevent further proliferation over a 24-hour period despite the ideal microbiological growth conditions. Opportunistic pathogenic microorganisms cause catheter-related infections by means of binding extraluminally or intraluminally to the catheter tube surface and subsequently developing into a biofilm. With the use of short-term catheters (<10 days) the majority of infections are often believed to originate extraluminally from the cutaneous layer. The results from our antimicrobial study suggest the inhibition of bacterial growth in close proximity to the tubing surface would support the use of NO as an effective antimicrobial agent in this setting.

### IN CONCLUSION

Addressing the important experimental challenges associated with the processing of MOFs into articles that are applicable across a wide range of uses is vital if we are to develop this fascinating class of materials so that they reach their potential for application. The identification of the processing parameters for successful extrusion of MOF-polymer composite tubing, combined with careful consideration of gas-loading and delivery conditions, has enabled us to demonstrate that the same composite can deliver the correct amount of nitric oxide to target the three most common complications in cardiovascular catheterisation. While this particular study is targeted at the development of medical devices, the materials science and engineering we have demonstrated is translatable across to other gas handling applications. As such, the practical advances demonstrated by the reported experimental design and characterisation will guide future developments in the field of MOF applications across a wide range of fields. This will be especially true when competition between different modes of adsorption, as is the case here, need to be taken into account. The manuscript also identifies new adsorption sites in the partially activated MOFs, which are not seen in fully activated materials. This indicates that developing new processes that are constrained in temperature that mean full activation is not possible will not necessarily compromise adsorption capacity of the MOF, despite there being a reduction in open metal sites.

### REFERENCES

1. McKinlay, A. et al. Exceptional Behavior over the Whole Adsorption-Storage-Delivery Cycle for NO in Porous Metal Organic Frameworks. J. Am. Chem. Soc. 130, 10440-10444 (2008)
2. Hinks, N. J., McKinlay, A. C., Xiao, B., Wheatley, P. S. & Morris, R. E. Metal organic frameworks as NO delivery materials for biological applications. Micropor. Mesopor. Mater. 129, 330-334 (2010).
3. Xiao, B., et al High-capacity hydrogen and nitric oxide adsorption and storage in a metal-organic framework. J Am Chem Soc 129, 1203-1209 (2007).
4. Duncan, M. J., et al Antibacterial efficacy from NO-releasing MOF-polymer films. Mater. Adv. 1, 2509-2519 (2020).
5. Frameworks for commercial success Nat. Chem 8, 987 (2016).
6. Yeskendir, B., Dacquin, J-P., Lorgouilloux, Y., Courtois, C., Royer, S., & Dhainaut, J., From metal-organic framework powders to shaped solids: recent developments and challenges. Mater Adv. 2, 7139-7186 (2021).
7. Li, Y., Wen, G., Li, J., Li, Q., Zhang, H., Tao, B., & Zhang, J. Synthesis and shaping of metal-organic frameworks: a review. Chem. Commun. 58, 11488-11506 (2022).
8. Wang, Z., Liu, L., Li, Z., Goyal, N., Du, T., He, J., & Li, G. K. Shaping of Metal-Organic Frameworks: A Review. Energy Fuels 36, 2927-2944 (2022).
9. Jia, S., Ji, D., Wang, L., Qin, X., & Ramakrishna S. Metal-Organic Framework Membranes: Advances, Fabrication, and Applications. Small Struct. 3, 2100222 (2022).
10. Harding, J. L., & Reynolds, M. M. Composite materials with embedded metal organic framework catalysts for nitric oxide release from bioavailable S-nitrosothiols. J. Mater. Chem. B 2, 2530-2535 (2014).
11. Kathuria, A., Abiad, M. G, & Auras, R. Toughening of poly(L-lactic acid) with Cu3BTC2 metal organic framework crystals. Polymer 54, 6679-6986, (2013).
12. Kathuria, A., Abiad, M. G, & Auras, R. Deterioration of metal-organic framework crystal structure during fabrication of poly(L-lactic acid) mixed-matrix membranes. Polym. Int. 62, 1144-1151 (2013).
13. Kathuria, A., Al-Ghamdi, S., Abiad, M. G. & Auras, R. The Influence of Cu3(BTC)2 metal organic framework on the permeability and perm-selectivity of PLLA-MOF mixed matrix membranes. J. Appl. Polym. 42764 (2015).
14. Kathuria, A., Abiad, M. G. & Auras, R. PLLA-ZIF-8 metal organic framework composites for potential use in food applications: Production, characterization and migration studies. Packag. Technol. Sci. 34, 393-400 (2021).
15. Sun, H., Xian, Y., Lai, Y., Zhang, H., Yu, B., Liu, Y., & Bhat, G. Evaluation of the Mercury Ions Adsorption Capacity of Copper-Based Metal-Organic Frameworks/poly(lactic acid) Composites. Polym-Plast Tech. Mat. 59(5), 505-516 (2020)
16. Dietzel, P. D. C, Johnsen, R. E., Blom, R. & Fjellvag, H. Structural Changes and Coordinatively Unsaturated Metal Atoms on Dehydration of Honeycomb Analogous Microporous Metal-Organic Frameworks. Chem. Eur. J. 14, 2389-2397 (2008).
17. Cattaneo, D., et al. Water Based Scale-up of CPO-27 Synthesis for Nitric Oxide Delivery. Dalton Trans. 45, 618-629 (2016).
18. Vornholt, S. M., et al. Controlled Synthesis of Large Single Crystals of Metal-Organic Framework CPO-27-Ni Prepared by a Modulation Approach: In situ Single-Crystal X-ray Diffraction Studies. Chem-Eur J 27, 8537-8546 (2021).
19. Hanikel, N., et al. of water structures in metal-organic frameworks for improved atmospheric water harvesting. Science 374, 454-459 (2021).

1. An article comprising at least a portion for releasing adsorbed Nitric Oxide (NO) gas, the portion comprising, consisting essentially of, or consisting of, a polymer/metal organic framework (MOF) composite material, wherein the MOF is partially activated and comprises NO gas bound to the MOF structure.

2. An article according to claim 1, wherein the article is a medical article for releasing a physiological beneficial amount of the adsorbed NO gas over a period of time into a body of a subject.

3. The article according to claim 1, wherein the NO gas is bound to at least water molecules bound to the MOF following partial activation, or both metal and water molecules present within pores of the MOF and optionally physisorbed NO that is bound within the polymer matrix.

4. The article according to claim 1, wherein the MOF is present in the composite material in the form of particles or powder, dispersed within the polymer.

5. The article according to claim 1, wherein the MOF is present in the composite material in an amount of between 0.1-12 wt %, 0.1/0.25-10 wt %, such as 0.1/0.25/0.5-5 wt %.

6. The article according to claim 1, wherein the MOF comprises a CPO-27-M framework, wherein M=Co, Fe, Mn, Mg, Ni, or Zn, such as Ni or Zn.

7. The article according to claim 1, wherein the polymer is a hydrophilic or hydrophobic polymer or copolymer.

8. The article according to claim 1, wherein the polymer is a thermoplastic polymer or copolymer, such as a thermoplastic elastomer.

9. The article according to claim 8, wherein the thermoplastic polymer is a polyether, polyurethane, polyamide, polyolefin, a fluoropolymer, polybutadiene, polyvinyl chloride, neoprene, or silicone polymer or co-polymer, comprising one or more of the identified polymers.

10. The article according to claim 9 wherein the polymer is a block copolymer comprising of rigid and soft blocks.

11. The article according to claim 10, wherein the block copolymer is a polyether/polyamide co-polymer (aka PEBA), such as a Pebax® or Vestamid® polymer.

12. The article according to claim 9, wherein the composite material is rigid, partially flexible, or flexible.

13. The article according to claim 1, wherein the composite material has been prepared by a melt process.

14. The article according to claim 1, wherein the composite material is in the form of a tube.

15. A medical article according to claim 14, when dependent on claim 2, wherein the tube is part of a medical catheter, cannula, or introducer sheath for insertion within a body, vessel, or organ of a subject, or is suitable for medical uses that do not require insertion into a body.

16. The medical article according to claim 15 wherein the tube is intended be retained within the body, vessel, or organ of a subject for a period of between 10 min, 20 min, or 30 min—14 days, such as 1 hour to 2 days, or 2 hours to 24 hour—1 week.

17. A method of adsorbing a NO gas within a polymer/MOF composite material, the method comprising placing the composite material under sub atmospheric pressure at a temperature below the melting point of the polymer, in order to partially activate the MOF and contacting the composite material with NO gas, such that NO gas binds to both metal and water molecules present within pores of the MOF.

18. The method according to claim 17, further comprising removing excess and/or weakly bound NO gas from the composite material by flushing the composite material under a reduced pressure (such as 10⁻² torr) for a few seconds (e.g. 2-30) with a dry inert gas, such as argon.

19. The method according to claim 17, further comprising purging the composite with a dry inert gas, such as argon in order to remove a portion of the bound NO gas, such as gas not removed by flushing alone, from the composite.

20. The method according to claim 19, wherein purging comprises placing the composite material under sub atmospheric pressure at an elevated temperature for at least one hour followed by one or more cycles of flushing the composite with a dry inert gas.

21. A medical article comprising a composite material obtained by the method according to claim 17.

22. A method of releasing a gas within a body of a subject, the method comprising inserting into the body of the subject a medical article according to claim 2, or claim 21.
